SEQUENCE LISTING (41 sequences)

SEQ ID NO: 1 - protein, 157 aa, Homo sapiens
  REGION 1..157: note = misc_feature - Human mature IL-18
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, Mus musculus
  REGION 1..157: note = misc_feature - Mouse mature IL-18
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 3 - skipped

SEQ ID NO: 4 - protein, 157 aa, synthetic construct
  REGION 1..157: note = Synthetic: Human IL-18 hCS
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 5 - protein, 157 aa, synthetic construct
  REGION 1..157: note = Synthetic: Human IL-18 L45C/E192C
YFGKLESKCSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNCD

SEQ ID NO: 6 - protein, 157 aa, synthetic construct
  REGION 1..157: note = Synthetic: Human IL-18 N50C
YFGKLESKLSVIRCLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLACEKERDLFKLILKKEDELGDRSIMFTVQNED

SEQ ID NO: 7 - protein, 157 aa, synthetic construct
  REGION 1..157: note = Synthetic: Human IL-18 L174C
YFGKLESKLSVIRNLNDQVLFIDQGNRPLFEDMTDSDSRDNAPRTIFIISMYKDSQPRGMAVTISVKSEKISTLSCENKIISFKEMNPPDNIKDTKSDIIFFQRSVPGHDNKMQFESSSYEGYFLASEKERDLFKLICKKEDELGDRSIMFTVQNED

SEQ ID NO: 8 - protein, 157 aa, synthetic construct
  REGION 1..157: note = Synthetic: Human IL-18 N50C/L174C
YFGKLESKLSVIRCLNDQVLFIDQGNRPLFEDMTDSDSRDNAPRTIFIISMYKDSQPRGMAVTISVKSEKISTLSCENKIISFKEMNPPDNIKDTKSDIIFFQRSVPGHDNKMQFESSSYEGYFLACEKERDLFKLICKKEDELGDRSIMFTVQNED

SEQ ID NO: 9 - protein, 157 aa, synthetic construct
  REGION 1..157: note = Synthetic: Human IL-18 Y37C/S91C
CFGKLESKLSVIRNLNDQVLFIDQGNRPLFEDMTDSDSRDNAPRTIFIISMYKDCQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 10 - protein, 157 aa, synthetic construct
  REGION 1..157: note = Synthetic: Human IL-18 F38C/I128C
YCGKLESKLSVIRNLNDQVLFIDQGNRPLFEDMTDSDSRDNAPRTIFIISMYKDSQPRGMAVTISVKSEKISTLSSENKIISFKEMNPPDNCKDTKSDIIFFQRSVPGHDNKMQFESSSYEGYFLASEKERDLFKLILKKEDELGDRSIMFTVQNED

SEQ ID NO: 11 - protein, 157 aa, synthetic construct
  REGION 1..157: note = Synthetic: Human IL-18 G39C/K89C
YFCKLESKLSVIRNLNDQVLFIDQGNRPLFEDMTDSDSRDNAPRTIFIISMYCDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 12 - protein, 157 aa, synthetic construct
  REGION 1..157: note = Synthetic: Human IL-18 S43C/S86C
YFGKLECKLSVIRNLNDQVLFIDQGNRPLFEDMTDSDSRDNAPRTIFIICMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 13 - protein, 157 aa, synthetic construct
  REGION 1..157: note = Synthetic: Human IL-18 S46C/V189C
YFGKLESKLCVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTCQNED

SEQ ID NO: 14 - protein, 157 aa, synthetic construct
  REGION 1..157: note = Synthetic: Human IL-18 S46C/I85C
YFGKLESKLCVIRNLNDQVLFIDQGNRPLFEDMTDSDSRDNAPRTIFICSMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 15 - protein, 157 aa, synthetic construct
  REGION 1..157: note = Synthetic: Human IL-18 V47C/Q190C
YFGKLESKLSCIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVCNED

SEQ ID NO: 16 - protein, 157 aa, synthetic construct
  REGION 1..157: note = Synthetic: Human IL-18 R49C/T188C
YFGKLESKLSVICNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFCVQNED

SEQ ID NO: 17 - protein, 157 aa, synthetic construct
  REGION 1..157: note = Synthetic: Human IL-18 N50C/Q54C
YFGKLESKLSVIRCLNDCVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 18 - protein, 157 aa, synthetic construct
  REGION 1..157: note = Synthetic: Human IL-18 F57C/T81C
YFGKLESKLSVIRNLNDQVLCIDQGNRPLFEDMTDSDSRDNAPRCIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 19 - protein, 157 aa, synthetic construct
  REGION 1..157: note = Synthetic: Human IL-18 D90C/A97C
YFGKLESKLSVIRNLNDQVLFIDQGNRPLFEDMTDSDSRDNAPRTIFIISMYKCSQPRGMCVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 20 - protein, 157 aa, synthetic construct
  REGION 1..157: note = Synthetic: Human IL-18 V98C/Q139C
YFGKLESKLSVIRNLNDQVLFIDQGNRPLFEDMTDSDSRDNAPRTIFIISMYKDSQPRGMACTISVKSEKISTLSSENKIISFKEMNPPDNIKDTKSDIIFFCRSVPGHDNKMQFESSSYEGYFLASEKERDLFKLILKKEDELGDRSIMFTVQNED

SEQ ID NO: 21 - protein, 157 aa, synthetic construct
  REGION 1..157: note = Synthetic: Human IL-18 T99C/P124C
YFGKLESKLSVIRNLNDQVLFIDQGNRPLFEDMTDSDSRDNAPRTIFIISMYKDSQPRGMAVCISVKSEKISTLSSENKIISFKEMNCPDNIKDTKSDIIFFQRSVPGHDNKMQFESSSYEGYFLASEKERDLFKLILKKEDELGDRSIMFTVQNED

SEQ ID NO: 22 - protein, 157 aa, synthetic construct
  REGION 1..157: note = Synthetic: Human IL-18 S101C/T109C
YFGKLESKLSVIRNLNDQVLFIDQGNRPLFEDMTDSDSRDNAPRTIFIISMYKDSQPRGMAVTICVKSEKISCLSSENKIISFKEMNPPDNIKDTKSDIIFFQRSVPGHDNKMQFESSSYEGYFLASEKERDLFKLILKKEDELGDRSIMFTVQNED

SEQ ID NO: 23 - protein, 157 aa, synthetic construct
  REGION 1..157: note = Synthetic: Human IL-18 I107C/N123C
YFGKLESKLSVIRNLNDQVLFIDQGNRPLFEDMTDSDSRDNAPRTIFIISMYKDSQPRGMAVTISVKSEKCSTLSSENKIISFKEMCPPDNIKDTKSDIIFFQRSVPGHDNKMQFESSSYEGYFLASEKERDLFKLILKKEDELGDRSIMFTVQNED

SEQ ID NO: 24 - protein, 157 aa, synthetic construct
  REGION 1..157: note = Synthetic: Human IL-18 R140C/Q150C
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQCSVPGHDNKMCFESSSYEGYFLASEKERDLFKLILKKEDELGDRSIMFTVQNED

SEQ ID NO: 25 - protein, 157 aa, synthetic construct
  REGION 1..157: note = Synthetic: Human IL-18 G158C/K176C
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYECYFLASEKERDLFKLILKCEDELGDRSIMFTVQNED

SEQ ID NO: 26 - protein, 157 aa, synthetic construct
  REGION 1..157: note = Synthetic: Human IL-18 F160C/I185C
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYCLASEKERDLFKLILKKEDELGDRSCMFTVQNED

SEQ ID NO: 27 - protein, 157 aa, synthetic construct
  REGION 1..157: note = Synthetic: Human IL-18 A162C/S184C
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLCSEKERDLFKLILKKEDELGDRCIMFTVQNED

SEQ ID NO: 28 - protein, 157 aa, synthetic construct
  REGION 1..157: note = Synthetic: Human IL-18 A162C/I185C
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLCSEKERDLFKLILKKEDELGDRSCMFTVQNED

SEQ ID NO: 29 - skipped

SEQ ID NO: 30 - protein, 157 aa, synthetic construct
  REGION 1..157: note = Synthetic: Mouse IL-18 mCS
NFGRLHSTTAVIRNINDQVLFVDKRQPVFEDMTDIDQSASEPQTRLIIYMYKDSEVRGLAVTLSVKDSKMSTLSSKNKIISFEEMDPPENIDDIQSDLIFFQKRVPGHNKMEFESSLYEGHFLASQKEDDAFKLILKKKDENGDKSVMFTLTNLHQS

SEQ ID NO: 31 - protein, 157 aa, synthetic construct
  REGION 1..157: note = Synthetic: Mouse IL-18 T44C/L189C
NFGRLHSTCAVIRNINDQVLFVDKRQPVFEDMTDIDQSASEPQTRLIIYMYKDSEVRGLAVTLSVKDSKMSTLSSKNKIISFEEMDPPENIDDIQSDLIFFQKRVPGHNKMEFESSLYEGHFLASQKEDDAFKLILKKKDENGDKSVMFTLTNCHQS

SEQ ID NO: 32 - protein, 157 aa, synthetic construct
  REGION 1..157: note = Synthetic: Mouse IL-18 N49C
NFGRLHSTTAVIRCINDQVLFVDKRQPVFEDMTDIDQSASEPQTRLIIYMYKDSEVRGLAVTLSVKDSKMSTLSSKNKIISFEEMDPPENIDDIQSDLIFFQKRVPGHNKMEFESSLYEGHFLACQKEDDAFKLILKKKDENGDKSVMFTLTNLHQS

SEQ ID NO: 33 - protein, 157 aa, synthetic construct
  REGION 1..157: note = Synthetic: Mouse IL-18 L171C
NFGRLHSTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLASQKEDDAFKLICKKKDENGDKSVMFTLTNLHQS

SEQ ID NO: 34 - protein, 157 aa, synthetic construct
  REGION 1..157: note = Synthetic: Mouse IL-18 N49C/L171C
NFGRLHSTTAVIRCINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLICKKKDENGDKSVMFTLTNLHQS

SEQ ID NO: 35 - protein, 311 aa, Homo sapiens
  REGION 1..311: note = misc_feature - Human IL-18Ralpha
AESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTR

SEQ ID NO: 36 - protein, 164 aa, Homo sapiens
  REGION 1..164: note = misc_feature - Human IL-18BP
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 37 - protein, 307 aa, Mus musculus
  REGION 1..307: note = misc_feature - Mouse IL-18Ralpha
KSCIHRSQIHVVEGEPFYLKPCGISAPVHRNETATMRWFKGSASHEYRELNNRSSPRVTFHDHTLEFWPVEMEDEGTYISQVGNDRRNWTLNVTKRNKHSCFSDKLVTSRDVEVNKSLHITCKNPNYEELIQDTWLYKNCKEISKTPRILKDAEFGDEGYYSCVFSVHHNGTRYNITKTVNITVIEGRSKVTPAILGPKCEKVGVELGKDVELNCSASLNKDDLFYWSIRKEDSSDPNVQEDRKETTTWISEGKLHASKILRFQKITENYLNVLYNCTVANEEAIDTKSFVLVRKEIPDIPGHVFTG

SEQ ID NO: 38 - protein, 165 aa, Mus musculus
  REGION 1..165: note = misc_feature - Mouse IL-18BP
TSAPQTTATVLTGSSKDPCSSWSPAVPTKQYPALDVIWPEKEVPLNGTLTLSCTACSRFPYFSILYWLGNGSFIEHLPGRLKEGHTSREHRNTSTWLHRALVLEELSPTLRSTNFSCLFVDPGQVAQYHIILAQLWDGLKTAPSPSQETLSSHSPVSRSAGPGVA

SEQ ID NO: 39 - protein, 398 aa, synthetic construct
  REGION 1..398: note = Synthetic: Human IL-18 A162C/I185C DR Fc
YFGKLESKLSVIRNLNDQVLFIDQGNRPLFEDMTDSDSRDNAPRTIFIISKYSDSLARGLAVTISVKSEKISTLSSENKIISFKEMNPPDNIKDTKSDIIFFQRDVPGHSRKMQFESSSYEGYFLCSEKERDLFKLILKKEDELGDRSCMFTVQNEDGGGGAGGGGAGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 399 aa, synthetic construct
  REGION 1..399: note = Synthetic: Mouse IL-18 T44C/L189C Fc
NFGRLHSTCAVIRNINDQVLFVDKRQPVFEDMTDIDQSASEPQTRLIIYMYKDSEVRGLAVTLSVKDSKMSTLSSKNKIISFEEMDPPENIDDIQSDLIFFQKRVPGHNKMEFESSLYEGHFLASQKEDDAFKLILKKKDENGDKSVMFTLTNCHQSGGGGAGGGGEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 41 - protein, 762 aa, synthetic construct
  REGION 1..762: note = Synthetic: Mouse IL-18 T44C/L189C MSA
NFGRLHSTCAVIRNINDQVLFVDKRQPVFEDMTDIDQSASEPQTRLIIYMYKDSEVRGLAVTLSVKDSKMSTLSSKNKIISFEEMDPPENIDDIQSDLIFFQKRVPGHNKMEFESSLYEGHFLASQKEDDAFKLILKKKDENGDKSVMFTLTNCHQSGGGENLYFQGHHHHHHHHGGGEAHKSEIAHRYNDLGEQHFKGLVLIAFSQYLQKSSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

(19) United States
(12) Patent Application Publication
(10) Pub. No.: US 2024/0409599 A1
(43) Pub. Date: Dec. 12, 2024

(54) Stabilized IL-18 Polypeptides and Uses Thereof

(71) Applicant: Genentech, Inc., South San Francisco, CA (US)

(72) Inventors: Travis William Bainbridge, Woodbridge, CA (US); Beyza Bulutoglu Baykara, Foster City, CA (US); Jonathan Thomas Sockolosky, South San Francisco, CA (US)

(73) Assignee: Genentech, Inc., South San Francisco, CA (US)

(21) Appl. No.: 18/669,898

(22) Filed: May 21, 2024

Related U.S. Application Data
(63) Continuation of application No. PCT/US2022/081529, filed on Dec. 14, 2022.
(60) Provisional application No. 63/289,948, filed on Dec. 15, 2021.

(51) Int. Cl. C07K 14/54; A61K 38/00; A61P 35/00; G01N 33/53; G01N 33/68

(52) U.S. Cl. CPC C07K 14/54; A61P 35/00; G01N 33/53; G01N 33/6869; A61K 38/00; C07K 2319/30; G01N 2333/54; G01N 2333/7155

(57) ABSTRACT

The invention provides stabilized IL-18 polypeptides and methods of making and using the same.

### CROSS-REFERENCE TO RELATED APPLICATIONS

This application is a continuation of International Application No. PCT/US2022/081529, filed Dec. 14, 2022, which claims the benefit of priority of U.S. Provisional Application No. 63/289,948, filed Dec. 15, 2021, each of which is incorporated by reference herein in its entirety for any purpose.

### SEQUENCE LISTING

The present application contains a Sequence Listing, which has been submitted electronically in XML format. Said XML copy, created on Apr. 5, 2023, is named “01146-0109-60US.xml” and is 57,543 bytes in size. The information in the electronic format of the sequence listing is incorporated herein by reference in its entirety.

### FIELD

The present invention relates to stabilized IL-18 polypeptides and methods of making and using the same.

### BACKGROUND

Interleukin 18 (IL-18) is a pro-inflammatory, IL-1 family cytokine. Like other IL-1 family members, IL-18 does not contain a signal peptide and is therefore not secreted in a manner typical of most soluble proteins, but rather uses an unconventional protein secretion pathway [Zhang et al., Cell, 2020]. Instead of a signal peptide, the amino terminus of IL-18 contains an inhibitory propeptide sequence, which keeps the cytokine in an inactive state until proteolytically cleaved by caspase 1 into the mature form [Tsutsumi et al., Sci Rep, 2019]. IL-18 has two cognate signaling coreceptors, IL-18Rα and IL-18Rβ, as well as a soluble “decoy” receptor, IL-18BP. The IL-18 signaling pathway is initiated by mature IL-18 binding first to IL-18Rα, the IL-18/IL-18Rα complex then recruits IL-18Rβ into a high-affinity heterotrimeric complex which signals through the intracellular toll/interleukin 1 receptor (TIR) domains of the receptors. In contrast, IL-18BP, which has homology with IL-18Rα, has a very strong affinity for the mature cytokine and can sterically block IL-18 from signaling through IL-18Rα/IL-18Rβ, functioning as a natural antagonist.

IL-18 signaling can promote effector T cell maturation and function, and can stimulate NK cells. There is emerging insight suggesting IL-18 signaling can drive anti-tumor immunity, both as a single agent therapy as well as in combination with checkpoint blockade agents. Clinical efficacy with the wild-type cytokine has been limited [Tarhini et al., Cancer, 2009], likely because it is inhibited by the relatively abundant IL-18 antagonist, IL-18BP. However, recent work has shown that IL-18 variants which are engineered to be resistant to IL-18BP provide a superior anti-tumor response in mouse models, in comparison to the WT cytokine [Zhou et al., Nature, 2020]. As a therapeutic, some features of IL-18 may pose a challenge, such as its short half-life and lability.

Recombinant IL-18 is most commonly produced in E. coli in low yields, either in its pro-form [Kirkpatrick et al., Protein Expression & Purification, 2003] or genetically fused with a stabilizing partner, such as SUMO [Kato et al., Nat Struct Biol, 2003; Krumm et al., Structural Biology Communications, 2015], which must be further processed by proteases after purification, for functional activity. There have been several clinical trials with IL-18, including the those by GSK [Tarhini et al., Cancer, 2009; Robertson et al., J Immunother, 2013] and by Simcha therapeutics (Clinicaltrials.gov identifier: NCT04787042), which have all used recombinant cytokine produced in E. coli. Recombinant protein production in mammalian host cells provides attractive advantages over E. coli, including low levels of endotoxin and the ability to produce more complex formats such as fusions with albumin or the Fc domain of IgG for in vivo half-life extension, which benefit from mammalian protein folding machinery for disulfide bond formation and glycosylation. Recombinant IL-18 is a challenging molecule to produce, especially in mammalian host cells, due in part to its unconventional secretion mechanism which lacks the use of a signal peptide, and the presence of an N-terminal inhibitory propeptide which is necessary to remove in order to produce the active cytokine [Dinarello et al., Front. Immunol., 2013]. Additionally, both human and murine IL-18 contain free cysteines, none of which form functional intramolecular disulfides and so they remain available for the formation of non-native, disulfide-linked intermolecular aggregates under non-reducing conditions. Rapid inactivation of the molecule was also shown due to oxidation of cysteine residues [Cohen et al., Nat Comm., 2015]. Furthermore, IL-18 has been reported to have a relatively low thermostability, particularly after removal of the propeptide [Tsutsumi et al., Sci Rep, 2019]. As such, the inherent molecular instability of IL-18 causes significant obstacles in the use of this molecule as a research reagent, and it may result in significant CMC liabilities for IL-18-based therapeutics.

There remains a need for stabilized IL-18. The invention described herein meets this need and provides other benefits.

### SUMMARY

The invention provides stabilized IL-18 proteins and methods of making and using the same.

Embodiment 1. A polypeptide comprising a modified human IL-18 polypeptide, wherein the amino acid sequence of the modified human IL-18 polypeptide is at least 80%, at least 85%, at least 90%, at least 91%, at least 92%, at least 93%, at least 94%, at least 95%, at least 96%, or at least 97% identical to the amino acid sequence of SEQ ID NO: 1, and wherein the modified human IL-18 polypeptide comprises at least one pair of cysteines that are capable of forming a disulfide bond.

Embodiment 2. The polypeptide of embodiment 1, wherein the modified human IL-18 polypeptide does not comprise free cysteines.

Embodiment 3. The polypeptide of embodiment 1 or embodiment 2, wherein the modified human IL-18 polypeptide comprises one or two pairs of cysteines, wherein each pair of cysteines forms a disulfide bond.

Embodiment 4. The polypeptide of any one of embodiments 1-3, wherein at least one, at least two, at least three, or all four cysteines in the amino acid sequence of SEQ ID NO: 1 are substituted with another amino acid.

Embodiment 5. The polypeptide of embodiment 4, wherein at least one, at least two, at least three, or all four cysteines in the amino acid sequence of SEQ ID NO: 1 are substituted with serine.

Embodiment 6. The polypeptide of any one of embodiments 1-5, wherein the modified human TL-18 polypeptide comprises one, two, three, or four of amino acid substitutions C74S, C104S, C112S, and/or C163S, wherein amino acid numbering is according to FIG. 4A.

Embodiment 7. The polypeptide of any one of embodiments 1-6, wherein the modified human IL-18 polypeptide comprises a set of amino acid substitutions selected from:
a) L45C and E192C;
b) Y37C and S91C;
c) S43C and S86C;
d) S46C and V189C;
e) S46C and I85C;
f) V47C and Q190C;
g) N50C;
h) N50C and L174C;
i) F57C and T81C;
j) D90C and A97C;
k) V98C and Q139C;
l) T99C and P124C;
m) S101C and T109C;
n) I107C and N123C;
o) R140C and Q150C; and
p) A162C and I185C;
wherein amino acid numbering is according to FIG. 4A.

Embodiment 8. A polypeptide comprising a modified human IL-18 polypeptide, wherein the amino acid sequence of the modified human IL-18 polypeptide is at least 80%, at least 85%, at least 90%, at least 91%, at least 92%, at least 93%, at least 94%, at least 95%, at least 96%, or at least 97% identical to the amino acid sequence of SEQ ID NO: 1, and wherein the modified human IL-18 polypeptide comprises a set of amino acid substitutions selected from:
a) L45C and E192C;
b) Y37C and S91C;
c) S43C and S86C;
d) S46C and V189C;
e) S46C and I85C;
f) V47C and Q190C;
g) N50C;
h) N50C and L174C;
i) F57C and T81C;
j) D90C and A97C;
k) V98C and Q139C;
l) T99C and P124C;
m) S101C and T109C;
n) I107C and N123C;
o) R140C and Q150C; and
p) A162C and I185C;
wherein amino acid numbering is according to FIG. 4A.

Embodiment 9. The polypeptide of any one of embodiments 1-6, wherein the modified human IL-18 polypeptide comprises a set of amino acid substitutions selected from:
a) L45C and E192C;
b) Y37C and S91C;
c) S43C and S86C;
d) S46C and V189C;
e) S46C and I85C;
f) V47C and Q190C;
g) F57C and T81C;
h) D90C and A97C;
i) V98C and Q139C;
j) T99C and P124C;
k) S101C and T109C;
1) I107C and N123C;
m) R140C and Q150C; and
n) A162C and I185C;
and comprises amino acid substitutions C74S, C104S, C112S, and C163S, wherein amino acid numbering is according to FIG. 4A.

Embodiment 10. The polypeptide of embodiment 7 or embodiment 8, wherein the modified human IL-18 polypeptide comprises a set of amino acid substitutions selected from:
a) N50C, C74S, C104S, and C112S; and
b) N50C, C74S, C104S, and L174C;
wherein amino acid numbering is according to FIG. 4A.

Embodiment 11. The polypeptide of any one of embodiments 1-10, wherein the amino acid sequence of the modified human IL-18 polypeptide is at least 90%, at least 95%, at least 96%, at least 97%, at least 98%, or at least 99% identical to an amino acid sequence selected from SEQ ID NOs: 5, 6, 8, 9, 12, 13, 15, 18, 19-24, and 27.

Embodiment 12. The polypeptide of any one of embodiments 1-10, wherein the modified human IL-18 polypeptide comprises an amino acid sequence selected from SEQ ID NOs: 5, 6, 8, 9, 12, 13, 15, 18, 19-24, and 27.

Embodiment 13. The polypeptide of any one of embodiments 1-12, wherein the polypeptide binds IL-18Rα.

Embodiment 14. The polypeptide of embodiment 11, wherein the polypeptide binds to IL-18Rα with an affinity of less than 100 nM, or less than 50 nM, or less than 30 nM, or less than 20 nM, less than 10 nM, between 0.1 nM and 100 nM, or between 1 nM and 100 nM, as measured by surface plasmon resonance.

Embodiment 15. The polypeptide of any one of embodiments 1-12, wherein the polypeptide binds with significantly reduced affinity to IL-18Rα compared to wild-type IL-18, or does not detectably bind IL-18Rα.

Embodiment 16. The polypeptide of embodiment 15, wherein the polypeptide binds to IL-18Rα with an affinity of greater than 50 nM, greater than 60 nM, greater than 70 nM, greater than 80 nM, greater than 90 nM, greater than 100 nM, between 50 nM and 1 mM, between 60 nM and 1 mM, between 70 nM and 1 mM, between 80 nM and 1 mM.

Embodiment 17. The polypeptide of embodiment 15, wherein the polypeptide shows no detectable binding to IL-18Rα up to 81 nM, as measured by surface plasmon resonance.

Embodiment 18. The polypeptide of any one of embodiments 1-17, wherein the polypeptide binds to IL-18BP.

Embodiment 19. The polypeptide of embodiment 18, wherein the polypeptide binds to IL-18BP with an affinity of less than 1 nM, less than 100 pM, or less than 50 pM, or less than 30 pM, or less than 20 pM, less than 10 pM, between 1 fM and 1 nM, between 10 fM and 1 nM, between 1 fM and 100 pM, between 10 fM and 100 pM, between 1 fM and 50 pM, between 10 fM and 50 pM, between 1 fM and 30 pM, or between 10 fM and 30 pM, as measured by surface plasmon resonance.

Embodiment 20. The polypeptide of any one of embodiments 1-19, wherein the polypeptide induces signaling through the IL-18 receptor in a reporter assay with an EC50 of less than 1 nM, less than 800 pM, less than 700 pM, less than 600 pM, less than 500 pM, less than 400 pM, less than 300 pM, less than 200 pM, less than 100 pM, between 1 pM and 1 nM, between 1 pM and 800 pM, between 1 pM and 500 pM, or between 1 pM and 300 pM.

Embodiment 21. The polypeptide of any one of embodiments 1-20, wherein the polypeptide induces IFNγ expression in human lymphocytes in vitro.

Embodiment 22. The polypeptide of embodiment 21, wherein the lymphocytes are T cells or NK cells.

Embodiment 23. The polypeptide of embodiment 21 or embodiment 22, wherein the polypeptide induces IFNγ expression in human T cells in vitro with an EC50 of less than 1 nM, less than 800 pM, less than 700 pM, less than 600 pM, less than 500 pM, less than 400 pM, less than 300 pM, less than 200 pM, less than 100 pM, between 1 pM and 1 nM, between 1 pM and 800 pM, between 1 pM and 500 pM, or between 1 pM and 300 pM.

Embodiment 24. The polypeptide of any one of embodiments 1-23, wherein the polypeptide induces IFNγ expression in human lymphocytes in vitro to a substantially reduced extent than wild-type human IL-18.

Embodiment 25. The polypeptide of embodiment 24, wherein the lymphocytes or T cells or NK cells.

Embodiment 26. The polypeptide of any one of the preceding embodiments, wherein the modified human IL-18 polypeptide further comprises at least one, at least two, at least three, at least four, at least five, or at least six substitutions at a position selected from Y37, L41, K44, M87, K89, S91, Q92, P93, G95, M96, E113, Q139, S141, D146, N147, M149, V189, and N191, wherein amino acid numbering is according to FIG. 4A.

Embodiment 27. The polypeptide of embodiment 26, wherein the modified human IL-18 polypeptide further comprises at least one, at least two, at least three, at least four, at least five, or at least six substitutions selected from Y37H, Y37R, L41H, L41I, L41Y, K44Q, K44R, M87T, M87K, M87D, M87N, M87E, M87R, K89R, K89G, K89S, K89T, S91K, S91R, Q92E, Q92A, Q92R, Q92V, Q92G, Q92K, Q92L, P93L, P93G, P93A, P93K, G95T, G95A, M96K, M96Q, M96R, M96L, E113D, Q139E, Q139K, Q139P, Q139A, Q139R, S141R, S141D, S141K, S141N, S141A, D146H, D146K, D146N, D146Q, D146E, D146S, D146G, N147H, N147Y, N147D, N147R, N147S, N147G, M149V, M149R, M149T, M149K, V189I, V189T, V189A, N191K, and N191H.

Embodiment 28. The polypeptide of any one of embodiments 1-27, wherein the modified human IL-18 polypeptide further comprises substitutions at positions M87, M96, S141, D146, and N147; or at positions M87, K89, Q92, S141, and N147, wherein amino acid numbering is according to FIG. 4A.

Embodiment 29. The polypeptide of embodiment 28, wherein the modified human IL-18 polypeptide further comprises substitutions (i) M87T or M87K; (ii) M96K or M96L; (iii) S141D, S141N, or S141A; (iv) D146K, D146N, D146S, or D146G; and (v) N147Y, N147Y, N147R, or N147G; or further comprises substitutions (i) M87K; (ii) K89G or K89S; (iii) Q92G, Q92R, or Q92L; (iv) D146N, D146S, or D146G; and (v) N147R or N147G.

Embodiment 30. The polypeptide of any one of embodiment 1-29, wherein the modified human IL-18 polypeptide further comprises at least one, at least two, at least three, at least four, at least five, or at least six substitutions at a position selected from Y37, L41, D53, E67, T70, D71, S72, D73, D76, N77, M87, Q91, M96, Q139, H145, M149, and R167, wherein amino acid numbering is according to FIG. 4A.

Embodiment 31. The polypeptide of embodiment 30, wherein the modified human IL-18 polypeptide further comprises at least one, at least two, at least three, at least four, at least five, or at least six substitutions selected from Y37D, Y37F, Y37H, Y37L, L41F, L41H, D53A, D53G, D53R, D53H, E67A, E67T, E67G, E67K, E67R, T70A, T70K, T70E, D71S, D71A, D71Y, S72N, S72K, S72R, D73P, D73A, D73R, D73H, D73L, D73V, D76Y, D76S, D76A, N77K, N77S, N77R, M87F, M87L, M87I, Q91H, M96L, M96F, M96I, Q139L, Q139I, H145A, H145P, H145D, M149L, M149I, M149F, and R167S.

Embodiment 32. The polypeptide of any one of embodiments 1-29, 30, and 31, wherein the modified human IL-18 polypeptide further comprises substitutions D53G, E66A, and either Q139L or Q139I.

Embodiment 33. The polypeptide of embodiment 32, wherein the modified human IL-18 polypeptide further comprises substitutions D71S and M87F.

Embodiment 34. The polypeptide of any one of embodiments 1-33, wherein the polypeptide comprises a fusion partner.

Embodiment 35. The polypeptide of embodiment 34, wherein the polypeptide has a longer half-life than the modified IL-18 polypeptide without the fusion partner.

Embodiment 36. The polypeptide of embodiment 34 or embodiment 35, wherein the fusion partner is an Fc domain, human serum albumin, or an antigen-binding domain.

Embodiment 37. The polypeptide of embodiment 36, wherein the Fc domain is an IgG1, IgG2, or IgG4 Fc domain.

Embodiment 38. The polypeptide of any one of embodiments 1-37, wherein the polypeptide does not comprise a fusion partner.

Embodiment 39. A conjugate comprising the polypeptide of any one of embodiments 1-38 and a conjugate moiety.

Embodiment 40. The conjugate of embodiment 39, wherein the conjugate moiety is a polymer, such as polyethylene glycol (PEG).

Embodiment 41. An isolated nucleic acid encoding the polypeptide of any of embodiments 1-38.

Embodiment 42. A host cell comprising the nucleic acid of embodiment 41.

Embodiment 43. A host cell that expresses the polypeptide of any one of embodiments 1-40.

Embodiment 44. A method of producing a polypeptide comprising a modified human IL-18 polypeptide, comprising culturing the host cell of embodiment 42 or embodiment 43 under conditions suitable for the expression of the polypeptide.

Embodiment 45. The method of embodiment 44, further comprising recovering the polypeptide from the host cell.

Embodiment 46. The method of embodiment 44 or embodiment 45, wherein the host cell is a eukaryotic host cell.

Embodiment 47. The method of embodiment 46, wherein the host cell is a mammalian host cell.

Embodiment 48. The method of embodiment 47, wherein the host cell is a CHO cell or a 293 cell.

Embodiment 49. A polypeptide produced by the method of any one of embodiments 44-48.

Embodiment 50. A pharmaceutical composition comprising the polypeptide of any one of embodiments 1-38 and 49, or the conjugate of embodiment 39 or embodiment 40, and a pharmaceutically acceptable carrier.

Embodiment 51. The pharmaceutical composition of embodiment 50, further comprising an additional therapeutic agent.

Embodiment 52. The pharmaceutical composition of embodiment 51, wherein the additional therapeutic agent is an immunooncology agent.

Embodiment 53. The pharmaceutical composition of embodiment 51 or embodiment 52, wherein the immunooncology agent is an immune checkpoint inhibitor or an agonist of an immune co-stimulatory molecule.

Embodiment 54. The pharmaceutical composition of any one of embodiments 51-53, wherein the additional therapeutic agent is an antibody that binds a tumor associated antigen; a CD28, OX40, GITR, CD137, CD27, CD40, ICOS, HVEM, NKG2D, MICA, 2B4, IL-2, IL-12, IL-27, IFNγ, IFNα, TNFα, IL-1, CDN, HMGB1, or TLR agonist; or a PD-1, PD-L1, CTLA-4, TIM-3, BTLA, VISTA, LAG-3, CD47, SIRPa, B7H4, CD96, TIGIT, CD226, prostaglandin, VEGF, endothelin B, IDO, arginase, MICA/MICB, TIM-3, IL-10, IL-4, or IL-13 antagonist.

Embodiment 55. The pharmaceutical composition of any one of embodiments 51-54, wherein the additional therapeutic agent is a PD-1 axis binding antagonist.

Embodiment 56. The pharmaceutical composition of embodiment 55, wherein the PD-1 axis antagonist is a PD-1 binding antagonist or a PDL1 binding antagonist.

Embodiment 57. The pharmaceutical composition of any one of embodiments 51-56, wherein the additional therapeutic agent is an antibody.

Embodiment 58. The pharmaceutical composition of any one of embodiments 51-56, wherein the additional therapeutic agent is selected from ipilimumab, pembrolizumab, nivolumab, atezolizumab, avelumab, durvalumab, utomilumab, urelumab, INBRX-105, GSK3359609, JTX-2011, TRX 518-001, MK-4166, BMS-986156, INCAGN01876, cusatuzumab, varlilumab, PF-0451860, MEDI0562/6469/6383, GSK3174998, BMS-986178, CP870893, APX005M, CA-170, mogamulizumab, MGD009, 8H9, TSR-022, MBG453, Sym023, oleclumab, relatlimab, IMP321 (eftilagimod alpha), LAG525, lirilumab, indoximod, epacadostat, tislelizumab, tiragolumab, BMS-986207, MTIG7192A, AB154, ciforadenant, M7824, galunisertib, TTI-621, evorpacept, magrolimab, oleclumab, poly-ICIC, lefitolimod, SD-101, DSP-0509, rintatolimod, CMP-001, NKTR-214, R06874281, THOR-707, CB-1158, LTX-315, or pegilodecakin.

Embodiment 59. The polypeptide of any one of embodiments 1-38 and 49, the conjugate of embodiment 39 or embodiment 40, or the pharmaceutical composition of any one of embodiments 50-58 for use as a medicament.

Embodiment 60. The polypeptide of any one of embodiments 1-38 and 49, the conjugate of embodiment 39 or embodiment 40, or the pharmaceutical composition of any one of embodiments 50-58 for use in treating cancer.

Embodiment 61. The polypeptide or pharmaceutical composition for use of embodiment 60, wherein the cancer is a carcinoma, lymphoma, blastoma, sarcoma, or leukemia.

Embodiment 62. The polypeptide or pharmaceutical composition for use of embodiment 60 or embodiment 61, wherein the cancer is adenocarcinoma (e.g., colorectal adenocarcinoma, gastric adenocarcinoma, or pancreatic adenocarcinoma), which may be metastatic adenocarcinomas (e.g., metastatic colorectal adenocarcinoma, metastatic gastric adenocarcinoma, or metastatic pancreatic adenocarcinoma), esophageal cancer, gastric or stomach cancer, small intestine cancer, large intestine cancer, small cell lung cancer, glioblastoma, neuroblastoma, melanoma, breast carcinoma, gastric cancer, colorectal cancer (CRC), hepatocellular carcinoma, breast cancer, rectal cancer, non-small cell lung cancer, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, multiple myeloma, mature B-Cell cancer excluding Hodgkin's Lymphoma but including germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, or B-cell lymphoma, unclassifiable, with features intermediate between diffuse large B-cell lymphoma and classical Hodgkin lymphoma.

Embodiment 63. Use of the polypeptide of any one of embodiments 1-38 and 49, the conjugate of embodiment 39 or embodiment 40, or the pharmaceutical composition of any one of embodiments 50-58 in the manufacture of a medicament for treating cancer.

Embodiment 64. The use of embodiment 63, wherein the cancer is a carcinoma, adenocarcinoma, lymphoma, blastoma, sarcoma, or leukemia.

Embodiment 65. The use of embodiment 63 or embodiment 64, wherein the cancer is adenocarcinoma (e.g., colorectal adenocarcinoma, gastric adenocarcinoma, or pancreatic adenocarcinoma), which may be metastatic adenocarcinomas (e.g., metastatic colorectal adenocarcinoma, metastatic gastric adenocarcinoma, or metastatic pancreatic adenocarcinoma), esophageal cancer, gastric or stomach cancer, small intestine cancer, large intestine cancer, small cell lung cancer, glioblastoma, neuroblastoma, melanoma, breast carcinoma, gastric cancer, colorectal cancer (CRC), hepatocellular carcinoma, breast cancer, rectal cancer, non-small cell lung cancer, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, multiple myeloma, mature B-Cell cancer excluding Hodgkin's Lymphoma but including germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, or B-cell lymphoma, unclassifiable, with features intermediate between diffuse large B-cell lymphoma and classical Hodgkin lymphoma.

Embodiment 66. A method of treating subject with cancer, comprising administering to the subject an effective amount of the polypeptide of any one of embodiments 1-38 and 49, the conjugate of embodiment 39 or embodiment 40, or the pharmaceutical composition of embodiment 50.

Embodiment 67. The method of embodiment 66, wherein the cancer is a carcinoma, adenocarcinoma, lymphoma, blastoma, sarcoma, or leukemia.

Embodiment 68. The method of embodiment 66 or embodiment 67, wherein the cancer is adenocarcinoma (e.g., colorectal adenocarcinoma, gastric adenocarcinoma, or pancreatic adenocarcinoma), which may be metastatic adenocarcinomas (e.g., metastatic colorectal adenocarcinoma, metastatic gastric adenocarcinoma, or metastatic pancreatic adenocarcinoma), esophageal cancer, gastric or stomach cancer, small intestine cancer, large intestine cancer, small cell lung cancer, glioblastoma, neuroblastoma, melanoma, breast carcinoma, gastric cancer, colorectal cancer (CRC), hepatocellular carcinoma, breast cancer, rectal cancer, non-small cell lung cancer, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, multiple myeloma, mature B-Cell cancer excluding Hodgkin's Lymphoma but including germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, or B-cell lymphoma, unclassifiable, with features intermediate between diffuse large B-cell lymphoma and classical Hodgkin lymphoma.

Embodiment 69. The method of any one of embodiments 66-68, comprising administering an additional therapeutic agent to the subject.

Embodiment 70. The method of embodiment 69, wherein the additional therapeutic agent is an immunooncology agent or a chemotherapeutic agent.

Embodiment 71. The method of embodiment 70, wherein the immunooncology agent is an immune checkpoint inhibitor or an agonist of an immune co-stimulatory molecule.

Embodiment 72. The method of any one of embodiments 69-71, wherein the additional therapeutic agent is an antibody that binds a tumor associated antigen; a CD28, OX40, GITR, CD137, CD27, CD40, ICOS, HVEM, NKG2D, MICA, 2B4, IL-2, IL-12, IL-27, IFNγ, IFNα, TNFα, IL-1, CDN, HMGB1, or TLR agonist; or a PD-1, PD-L1, CTLA-4, TIM-3, BTLA, VISTA, LAG-3, CD47, SIRPa, B7H4, CD96, TIGIT, CD226, prostaglandin, VEGF, endothelin B, IDO, arginase, MICA/MICB, TIM-3, IL-10, IL-4, or IL-13 antagonist.

Embodiment 73. The method of any one of embodiments 69-72, wherein the additional therapeutic agent is a PD-1 axis binding antagonist.

Embodiment 74. The method of embodiment 73, wherein the PD-1 axis antagonist is a PD-1 binding antagonist or a PDL1 binding antagonist.

Embodiment 75. The method of any one of embodiments 69-74, wherein the additional therapeutic agent is an antibody.

Embodiment 76. The method of any one of embodiments 69-75, wherein the additional therapeutic agent is selected from ipilimumab, pembrolizumab, nivolumab, atezolizumab, avelumab, durvalumab, utomilumab, urelumab, INBRX-105, GSK3359609, JTX-2011, TRX 518-001, MK-4166, BMS-986156, INCAGN01876, cusatuzumab, varlilumab, PF-0451860, MED10562/6469/6383, GSK3174998, BMS-986178, CP870893, APX005M, CA-170, mogamulizumab, MGD009, 8H9, TSR-022, MBG453, Sym023, oleclumab, relatlimab, IMP321 (eftilagimod alpha), LAG525, lirilumab, indoximod, epacadostat, tislelizumab, BMS-986207, MTIG7192A, AB154, ciforadenant, M7824, galunisertib, TTI-621, evorpacept, magrolimab, oleclumab, poly-ICIC, lefitolimod, SD-101, DSP-0509, rintatolimod, CMP-001, NKTR-214, R06874281, THOR-707, CB-1158, LTX-315, pegilodecakin.

Embodiment 77. A method of activating the IL-18 receptor on a cell, comprising contacting the cell with the polypeptide of any one of embodiments 1-38 and 49 or the conjugate of embodiment 39 or embodiment 40.

Embodiment 78. A method of inducing IFNγ expression in a lymphocyte, comprising contacting the lymphocyte with the polypeptide of any one of embodiments 1-38 and 49 or the conjugate of embodiment 39 or embodiment 40.

Embodiment 79. A method of activating a lymphocyte, comprising contacting the lymphocyte with the polypeptide of any one of embodiments 1-38 and 49 or the conjugate of embodiment 39 or embodiment 40.

Embodiment 80. The method of embodiment 78 or embodiment 79, wherein the lymphocyte is a T cell or a NK cell.

Embodiment 81. The method of any one of embodiments 77-80, wherein the cell or lymphocyte is in vitro.

Embodiment 82. The method of any one of embodiments 77-80, wherein the cell or lymphocyte is in vivo.

Embodiment 83. A method of improving the stability of a polypeptide comprising a human IL-18 amino acid sequence, comprising introducing at least one pair of cysteines that form a disulfide bond into the IL-18 amino acid sequence, to make a polypeptide comprising a modified human IL-18 polypeptide.

Embodiment 84. The method of embodiment 83, wherein the modified human IL-18 polypeptide does not comprise free cysteines.

Embodiment 85. The method of embodiment 83 or embodiment 84, wherein the modified human IL-18 polypeptide comprises one or two pairs of cysteines, wherein each pair of cysteines forms a disulfide bond.

Embodiment 86. The method of any one of embodiments 83-85, wherein at least one, at least two, at least three, or all four cysteines in the amino acid sequence of SEQ ID NO: 1 are substituted with another amino acid.

Embodiment 87. The method of embodiment 86, wherein at least one, at least two, at least three, or all four cysteines in the amino acid sequence of SEQ ID NO: 1 are substituted with serine.

Embodiment 88. The method of any one of embodiments 83-87, wherein the modified human TL-18 polypeptide comprises one, two, three, or four of amino acid substitutions C74S, C104S, C112S, and/or C163S, wherein amino acid numbering is according to FIG. 4A.

Embodiment 89. A method of detecting IL-18BP in a sample, comprising contacting the sample with a polypeptide of any one of embodiments 1-38 and 49, and detecting binding of the polypeptide to IL-18BP.

Embodiment 90. A method of detecting IL-18Rα in a sample, comprising contacting the sample with a polypeptide of any one of embodiments 1-38 and 49, and detecting binding of the polypeptide to IL-18Rα.

Embodiment 91. The method of embodiment 89 or embodiment 90, wherein the polypeptide comprises a detectable label.

### BRIEF DESCRIPTION OF THE FIGURES

FIG. 1A-1B show IL-18 constructs for mammalian production. FIG. 1A shows human and mouse IL-18 constructs. The human IL-18 constructs contain mature human IL-18, followed sequentially by a TEV protease site, a poly-histidine tag and a monomeric human Fc.

The mouse IL-18 constructs have a similar design, but with mouse serum albumin (MSA) at the C-terminus instead of an Fc. FIG. 1B shows a wild-type, mature IL-18 construct for E. coli production. A poly-histidine tagged small ubiquitin related modifier (SUMO) was fused to the N-terminus of mature human or mouse IL-18. Cleavage by SUMO protease results in a tag-free, mature IL-18 with a clean N-terminus.

FIG. 2A-2Z show increased yield and decreased aggregation of several novel disulfide variants of IL-18, relative to WT IL-18 via analytical size exclusion chromatograms and SDS-PAGE. Human IL-18 variants were expressed as monomeric human Fc fusions and mouse IL-18 variants were expressed as mouse albumin fusions.

FIG. 3A-3B show dose-response curves of IL-18 variants in a primary T-cell potency assay. FIG. 3A shows dose-response curves of human IL-18 variants in a primary human T-cell potency assay. FIG. 3B shows dose-response curves of mouse IL-18 variants in a primary mouse T-cell potency assay.

FIG. 4A-4B show the sequence of mature human IL-18 (4A; SEQ ID NO: 1) and mature mouse IL-18 (4B; SEQ ID NO: 2), including the amino acid numbering used herein.

FIG. 5A-5B show efficacy of mouse IL-18 T44C/L189C Fc (“dsIL-18Fc”) in an MC38 colon cancer model. FIG. 5A shows efficacy of dsIL-18Fc dosed at 0.1 mg/kg, 1 mg/kg, and 5 mg/kg twice weekly for five doses, alone (top row: second, third, and fourth graphs), and in combination with anti-PD-L1 antibody at 10 mg/kg (bottom row: second, third, and fourth graphs). Efficacy of control antibody at 10 mg/kg, alone (top row: first graph) and in combination with PD-L1 (bottom row: first graph) is also shown. The bold dashed line in each graph shows the control group (control antibody alone) for comparison, and the bold solid line in each graph is the group fit. The narrower lines show the data for each animal. FIG. 5B shows efficacy of WT IL-18 in the same model (right graph). The bold dashed line in each graph shows the control group (control antibody alone) for comparison, and the bold solid line in each graph is the group fit. The narrower lines show the data for each animal.

FIG. 6A-6B show backbone traces from the crystal structures of the human L45C/E192C variant (6A) and human A162C/1185C variant (6B). The novel disulfide in each structure is shown and labeled.

### DETAILED DESCRIPTION

IL-18 is a potent, immune-stimulating cytokine and despite early failures to demonstrate anti-tumor efficacy in clinical trials, more recent work suggests that circumvention of IL-18BP, a native IL-18 antagonist, has the potential to drastically improve the therapeutic response to the cytokine. Besides the IL-18BP hurtle, recombinant mature IL-18 is also rather labile and difficult to produce, even at a research scale. Additionally, relative to a traditional IgG-based therapeutic, recombinant IL-18 has a very short half-life of less than two days [Robertson et al., Clin Cancer Res, 2006]. Success of an IL-18 based therapeutic could be greatly enhanced by addressing these shortcomings.

IL-18-based therapeutics used in clinical trials have typically been produced in E. coli. The ability to generate large quantities of a protein therapeutic in a mammalian host greatly simplifies the production and purification processes, while also enabling more complex designs that incorporate features which require mammalian-specific post-translational modifications and protein folding mechanisms. For example, fusion of an IL-18 therapeutic to albumin or the Fc domain of an IgG could greatly improve the pharmacokinetic and/or pharmacodynamic properties of the molecule. Another approach which is becoming increasingly common is to add a targeting module, such as a Fab or VHH, in addition to the half-life extending module. These approaches all benefit from mammalian host production. We have demonstrated here that stabilization of IL-18 through engineered disulfides indeed enables high yields of stable molecules, including both Fc and albumin fusions. In addition, two variants we evaluated (N50C & N50C/L174C) demonstrated the unexpected property of having highly attenuated binding to IL-18Rα and significantly reduced potency in a cell-based assay, yet they maintained a strong affinity for IL-18BP. This unique property could be exploited to produce a molecular trap for the circulating IL-18BP antagonist, thereby enhancing IL-18 signaling.

Here, we engineered novel, non-native disulfide bonds into human and murine IL-18. Our IL-18 disulfide-stabilized IL-18 variants were successfully expressed in mammalian host cells and secreted to significantly higher yields and lower aggregation levels compared to E. coli-produced WT IL-18.

Additionally, several variants demonstrate improved thermal stability relative to WT, under non-reducing conditions. While we were unable to detect binding of two of the variants to IL-18Rα, the IL-18 variants generally have similar affinities to both IL-18Rα and IL-18BP, as determined by SPR. Finally, similar to the SPR results, most of the variants exhibit similar potencies to WT in a cell-based reporter assay. Together, these results suggest that the introduction of certain disulfide bonds into IL-18 can improve its drug-like properties as a therapeutic and can ease the production of the recombinant cytokine, including production of fusion proteins.

### I. Definitions

“Affinity” refers to the strength of the sum total of noncovalent interactions between a single binding site of a molecule (e.g., a protein) and its binding partner (e.g., a receptor). Unless indicated otherwise, as used herein, “binding affinity” refers to intrinsic binding affinity which reflects a 1:1 interaction between members of a binding pair (e.g., protein and receptor). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

The term “cancer” refers to the physiological condition in mammals that is typically characterized by unregulated cell growth. Examples of cancer include but are not limited to, adenocarcinoma (e.g., colorectal adenocarcinoma, gastric adenocarcinoma, or pancreatic adenocarcinoma), which may be metastatic adenocarcinoma (e.g., metastatic colorectal adenocarcinoma, metastatic gastric adenocarcinoma, or metastatic pancreatic adenocarcinoma), carcinoma, lymphoma, blastoma, sarcoma, and leukemia or lymphoid malignancies. More particular examples of such cancers include, but are not limited to, esophageal cancer, small intestine cancer, large intestine cancer, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases.

A “chemotherapeutic agent” refers to a chemical compound useful in the treatment of cancer. Examples of chemotherapeutic agents include alkylating agents such as thiotepa, cyclosphosphamide (CYTOXAN®), temozolomide (Methazolastone®, Temodar®), treosultan, and bendamustine hydrochloride (Treanda®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), irinotecan liposome injection (Onivyde®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gammalI and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine, cladribine (Leustat®) and nelarabine (Arranon®); pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), cabazitaxel (Jevtana®), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vincristine sulfate liposome (Marqibo®), vindesine (ELDISINE®, FILDESIN®), vinflunine (Javlor®) and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); sorafenib (e.g. Nexavar®); SU-11248 (sunitinib, SUTENT®, Pfizer); perifosine, COX-2 inhibitor (e.g. celecoxib or etoricoxib), proteosome inhibitor (e.g. PS341) such as carfilzomib (Kyprolis®) and ixazomib citrate (Ninlaro®); bortezomib (VELCADE®); CCI-779; tipifarnib (R11577); orafenib, ABT510; Bcl-2 inhibitor such as oblimersen sodium (GENASENSE®) and venetoclax (Venclexta®); pixantrone; EGFR inhibitors (see definition below) such as gefitinib (Iressa®); tyrosine kinase inhibitors (see definition below) such as bosutinib (Bosulif®), cabozantinib-s-malate (Cabometyx®, Cometriq®), afatinib dimaleate (Gilotrif®), imatinib mesylate (Gleevec®), ponatinib hydrochloride (Iclusig®); axitinib (Inlyta®), ibrutinib (Imbruvica®), sorafenib tosylate (Nexavar®), dasatinib (Sprycel®), osimertinib (Tagrisso®), erlotinib hydrochloride (Tarceva®), nilotinib (Tasigna®), lapatinib ditosylate (Tykerb®), crizotinib (Xalkori®) and pazopanib hydrochloride (Votrient®); serine-threonine kinase inhibitors such as rapamycin (sirolimus, RAPAMUNE®), everolimus (Afinitor®), and temsirolimus (Torisel®); farnesyltransferase inhibitors such as lonafarnib (SCH 6636, SARASAR™); NK1 receptor antagonist such as netupitant, and rolapitant hydrochloride (Varubi®); imiquimod (Aldara®); anaplastic lymphoma kinase (ALK) inhibitor such as alectinib (Alecensa®) and ceritinib (Zykadia®); histone deacetylase inhibitors such as belinostat (Beleodaq®) and vorinostat (Zolinza®); purine nucleoside antimetabolite such as clofarabine; mitogen-activated protein kinase kinase (MEK) inhibitors such as cobimetinib (Cotellic®) and trametinib (Mekinist®); nucleic acid synthesis inhibitors such as decitabine (Dacogen®); Hedgehog signaling pathway inhibitor such as vismodegib (Erivedge®) and sonidegib (Odomzo®); histone deacetylase inhibitor such as panobinostat (Farydak®); antifolate such as pralatrexate (Folotyn®), raltitrexed, and pemetrexed disodium (Alimta®); mitotic inhibitor such as eribulin mesylate (Halaven®); inhibitor of the cyclin-dependent kinases such as palbociclib (Ibrance®); depsipeptide such as romidepsin (Istodax®); epothilone B analog such as ixabepilone (Ixempra®); Janus kinase inhibitor such as ruxolitinib phosphate (Jakafi®); multiple kinase inhibitor such as lenvatinib mesylate (Lenvima®), vandetanib (Caprelsa®), regorafenib (Stivarga®), nintedanib (Vargatef®); nucleoside analog such as trifluridine; thymidine phosphorylase inhibitor such as tipiracil hydrochloride; PARP inhibitor such as olaparib (Lynparza®); thalidomide (Stivarga®, Thalomid®) and its derivative such as pomalidomide (Pomalyst®) and lenalidomide (Revlimid®); synthetic corticosteroid such as prednisone; analog of somatostatin such as lanreotide acetate (Somatuline®); protein translation inhibitor such as omacetaxine mepesuccinate (Synribo®); inhibitor of the associated enzyme B-Raf such as dabrafenib (Tafinlar®) and vemurafenib (Zelboraf®); arsenic trioxide (Trisenox®); uridine triacetate (Vistogard®); radium 223 dichloride (Xofigo®); trabectedin (Yondelis), phosphoinositide 3-kinase inhibitor such as idelalisib (Zydelig®); milfamurtide (Mepact®); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; gonadotropin-releasing hormone (GnRH) antagonist such as degarelix, leuproprelin, and triptorelin; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrozole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releasing hormone agonists, including leuprolide (LUPRON® and ELIGARD®), goserelin, buserelin, histrelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; dexamethasone; onapristone; anti-progesterones; estrogen receptor down-regulators (ERDs); anti-androgens such as flutamide, nilutamide (e.g. Nilandron®), abiraterone acetate (Zytiga®), cyproterone acetate (Cyprostat®), enzalutamide (Xtandi®) and bicalutamide; and pharmaceutically acceptable salts, acids or derivatives of any of the above; as well as combinations of two or more of the above.

An “effective amount” of an agent, e.g., a pharmaceutical composition, refers to an amount effective, at dosages and for periods of time necessary, to achieve the desired therapeutic or prophylactic result.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “IL-18”, as used herein, refers to any native IL-18 from any vertebrate source, including mammals such as primates (e.g., humans) and rodents (e.g., mice and rats), unless otherwise indicated. The term encompasses “full-length”, unprocessed IL-18 as well as any form of IL-18 that results from processing in the cell. The term also encompasses naturally occurring variants of IL-18, e.g., splice variants or allelic variants. The amino acid sequence of an exemplary mature human IL-18 is shown as amino acids 37-193 of UniProtKB/Swiss-Prot: Q14116 and in SEQ ID NO: 1 herein. The amino acid of an exemplary mature mouse IL-18 is shown as amino acids 36-192 of UniProt: P70380 and in SEQ ID NO: 2 herein.

The term “stabilized IL-18 polypeptide” refers to a polypeptide comprising a modified IL-18 polypeptide that has been modified to contain at least one pair of cysteines that are capable of forming a disulfide bond. A stabilized IL-18 polypeptide may comprise additional amino acid sequence in addition to the modified IL-18 polypeptide, such as, for example, a fusion partner.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” polypeptide is one which has been separated from a component of its natural environment. In some aspects, a polypeptide is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of a polypeptide of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the polypeptide in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth.

Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, polypeptides of the invention are used to delay development of a disease or to slow the progression of a disease.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Compositions and Methods

In some aspects, the invention is based, in part, on stabilized IL-18 polypeptides. In some embodiments, the stabilized IL-18 polypeptides comprise at least one disulfide bond. The stabilized IL-18 polypeptides are useful, e.g., for the treatment of cancer, infectious diseases, and inflammation.

### A. Exemplary Stabilized IL-18 Polypeptides

In some aspects, the invention provides stabilized IL-18 polypeptides. In some embodiments, a stabilized IL-18 polypeptide binds to IL-18Rα. In some embodiments, a stabilized IL-18 polypeptide binds to IL-18Rα with an affinity of less than 100 nM, or less than 50 nM, or less than 30 nM, or less than 20 nM, or less than 10 nM, or between 0.1 nM and 100 nM, or between 1 nM and 100 nM. In some embodiments, a stabilized IL-18 polypeptide binds to IL-18Rα with significantly reduced affinity to IL-18Rα compared to wild-type IL-18, or does not detectably bind IL-18Rα. In some embodiments, a stabilized IL-18 polypeptide binds to IL-18Rα with an affinity of greater than 50 nM, greater than 60 nM, greater than 70 nM, greater than 80 nM, greater than 90 nM, greater than 100 nM, between 50 nM and 1 mM, between 60 nM and 1 mM, between 70 nM and 1 mM, between 80 nM and 1 mM. In some embodiments, affinity is measured by surface plasmon resonance. In some embodiments, a stabilized IL-18 polypeptide shows no detectable binding to IL-18Rα, such as no detectable binding up to 81 nM, as measured by surface plasmon resonance.

In some embodiments, a stabilized IL-18 polypeptide binds to IL-18BP. In some embodiments, a stabilized IL-18 polypeptide binds to IL-18BP with an affinity of less than 1 nM, less than 100 pM, or less than 50 pM, or less than 30 pM, or less than 20 pM, less than 10 pM, between 1 fM and 1 nM, between 10 fM and 1 nM, between 1 fM and 100 pM, between 10 fM and 100 pM, between 1 fM and 50 pM, between 10 fM and 50 pM, between 1 fM and 30 pM, or between 10 fM and 30 pM. In some embodiments, affinity is measured by surface plasmon resonance.

In some embodiments, a stabilized IL-18 polypeptide induces signaling through the IL-18 receptor. In some embodiments, a stabilized IL-18 polypeptide induces signaling through the IL-18 receptor with an EC50 of less than 1 nM, less than 800 pM, less than 700 pM, less than 600 pM, less than 500 pM, less than 400 pM, less than 300 pM, less than 200 pM, less than 100 pM, between 1 pM and 1 nM, between 1 pM and 800 pM, between 1 pM and 500 pM, or between 1 pM and 300 pM, for example, in a reporter assay in vitro. In some embodiments, a stabilized IL-18 polypeptide induces IFNγ expression in human lymphocytes, such as T cells or NK cells. In some embodiments, a stabilized IL-18 polypeptide induces IFNγ expression in human T cells in vitro with an EC50 of less than 1 nM, less than 800 pM, less than 700 pM, less than 600 pM, less than 500 pM, less than 400 pM, less than 300 pM, less than 200 pM, less than 100 pM, between 1 pM and 1 nM, between 1 pM and 800 pM, between 1 pM and 500 pM, or between 1 pM and 300 pM. In some embodiments, a stabilized IL-18 polypeptide provided herein induces IFNγ expression in human lymphocytes, such as T cells or NK cells, in vitro to a substantially reduced extent than wild-type human IL-18.

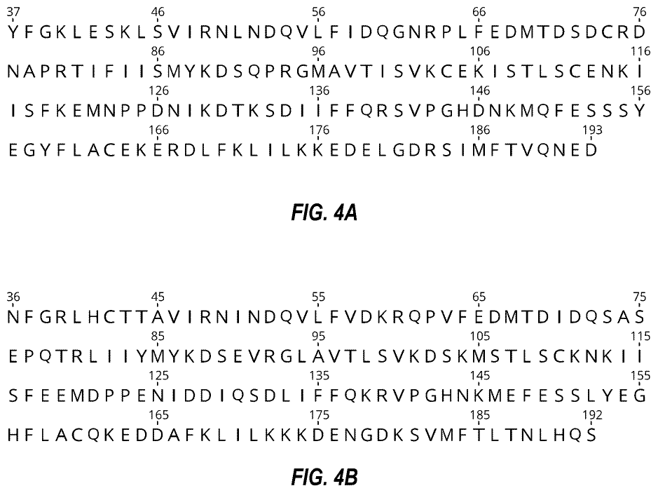

In various embodiments, a stabilized IL-18 polypeptide provided herein comprises a modified IL-18 polypeptide that has been engineered to comprise at least one pair of cysteines that are capable of forming a disulfide bond. In some embodiments, the modified IL-18 polypeptide has one or two pairs of cysteines. In some such embodiments, each pair of cysteines is capable of forming a disulfide bond. “Capable of forming a disulfide bond” means that the cysteines are in sufficient proximity and orientation to form and/or maintain a disulfide bond under appropriate conditions, such as physiological conditions. In some embodiments, the modified IL-18 polypeptide does not contain any free cysteines. In various embodiments, the modified IL-18 polypeptide is at least 80%, at least 85%, at least 90%, at least 91%, at least 92%, at least 93%, at least 94%, at least 95%, at least 96%, or at least 97% identical to the amino acid sequence of wild-type mature IL-18 (SEQ ID NO: 1). In some embodiments, any cysteines in the wild-type mature IL-18 amino acid sequence that are not part of the cysteine pairs capable of forming disulfide bonds are substituted with serines. Accordingly, in some embodiments, the modified IL-18 polypeptide comprises one, two, three, or four of amino acid substitutions C74S, C104S, C112S, and C163S, wherein amino acid numbering is according to FIG. 4A.

In some embodiments, a modified IL-18 polypeptide comprises a set of amino acid substitutions selected from:
a) L45C and E192C;
b) Y37C and S91C;
c) S43C and S86C;
d) S46C and V189C;
e) S46C and I85C;
f) V47C and Q190C;
g) N50C;
h) N50C and L174C;
i) F57C and T81C;
j) D90C and A97C;
k) V98C and Q139C;
1) T99C and P124C;
m) S101C and T109C;
n) I107C and N123C;
o) R140C and Q150C; and
p) A162C and I185C;
wherein amino acid numbering is according to FIG. 4A. In some such embodiments, the modified IL-18 polypeptide comprises one, two, three, or four of amino acid substitutions C74S, C104S, C112S, and C163S, wherein amino acid numbering is according to FIG. 4A.

In some embodiments, a modified IL-18 polypeptide comprises a set of amino acid substitutions selected from:
a) L45C and E192C;
b) Y37C and S91C;
c) S43C and S86C;
d) S46C and V189C;
e) S46C and I85C;
f) V47C and Q190C;
g) F57C and T81C;
h) D90C and A97C;
i) V98C and Q139C;
j) T99C and P124C;
k) S101C and T109C;
1) I107C and N123C;
m) R140C and Q150C; and
n) A162C and I185C;
wherein amino acid numbering is according to FIG. 4A. In some such embodiments, the modified IL-18 polypeptide comprises amino acid substitutions C74S, C104S, C112S, and C163S, wherein amino acid numbering is according to FIG. 4A.

In some embodiments, a modified IL-18 polypeptide comprises an amino acid sequence that is at least 90%, at least 95%, at least 96%, at least 97%, at least 98%, or at least 99% identical to an amino acid sequence selected from SEQ ID NOs: 5, 9, 12, 13, 15, 18, 19-24, and 27. In some embodiments, a modified human IL-18 polypeptide comprises an amino acid sequence selected from SEQ ID NOs: 5, 9, 12, 13, 15, 18, 19-24, and 27.

In some embodiments, a modified IL-18 polypeptide comprises substitution N50C, wherein amino acid numbering is according to FIG. 4A. In some embodiments, a modified IL-18 polypeptide comprises substitution L174C, wherein amino acid numbering is according to FIG. 4A. In some such embodiments, a modified IL-18 polypeptide comprises substitutions N50C, C74S, C104S, and C112S; or substitutions N50C, C74S, C104S, and L174C, wherein amino acid numbering is according to FIG. 4A. In some embodiments, N50C is capable of forming a disulfide bond with the native cysteine, C163. In some embodiments, L174C is capable of forming a disulfide bond with the native cysteine, C112.

In some embodiments, a modified IL-18 polypeptide comprises an amino acid sequence that is at least 90%, at least 95%, at least 96%, at least 97%, at least 98%, or at least 99% identical to an amino acid sequence selected from SEQ ID NOs: 6 and 8. In some embodiments, a modified human IL-18 polypeptide comprises an amino acid sequence selected from SEQ ID NOs: 6 and 8.

The modified IL-18 polypeptides provided herein may comprise one or more additional substitutions. Nonlimiting exemplary additional substitutions that may be included in a modified IL-18 polypeptide include those described in US2019/0070262. In some embodiments, the modified IL-18 polypeptide further comprises at least one, at least two, at least three, at least four, at least five, or at least six substitutions at a position selected from Y37, L41, K44, M87, K89, S91, Q92, P93, G95, M96, E113, Q139, S141, D146, N147, M149, V189, and N191, wherein amino acid numbering is according to FIG. 4A. In some embodiments, the modified IL-18 polypeptide further comprises at least one, at least two, at least three, at least four, at least five, or at least six substitutions selected from Y37H, Y37R, L41H, L41I, L41Y, K44Q, K44R, M87T, M87K, M87D, M87N, M87E, M87R, K89R, K89G, K89S, K89T, S91K, S91R, Q92E, Q92A, Q92R, Q92V, Q92G, Q92K, Q92L, P93L, P93G, P93A, P93K, G95T, G95A, M96K, M96Q, M96R, M96L, E113D, Q139E, Q139K, Q139P, Q139A, Q139R, S141R, S141D, S141K, S141N, S141A, D146H, D146K, D146N, D146Q, D146E, D146S, D146G, N147H, N147Y, N147D, N147R, N147S, N147G, M149V, M149R, M149T, M149K, V189I, V189T, V189A, N191K, and N191H.

In some embodiments, the modified IL-18 polypeptide further comprises substitutions at positions M87, M96, S141, D146, and N147; or at positions M87, K89, Q92, S141, and N147. In some such embodiments, the modified IL-18 polypeptide further comprises substitutions (i) M87T or M87K; (ii) M96K or M96L; (iii) S141D, S141N, or S141A; (iv) D146K, D146N, D146S, or D146G; and (v) N147Y, N147Y, N147R, or N147G; or further comprises substitutions (i) M87K; (ii) K89G or K89S; (iii) Q92G, Q92R, or Q92L; (iv) D146N, D146S, or D146G; and (v) N147R or N147G.

In some embodiments, the modified IL-18 polypeptide further comprises at least one, at least two, at least three, at least four, at least five, or at least six substitutions at a position selected from Y37, L41, D53, E67, T70, D71, S72, D73, D76, N77, M87, Q91, M96, Q139, H145, M149, and R167, wherein amino acid numbering is according to FIG. 4A. In some such embodiments, the modified human IL-18 polypeptide further comprises at least one, at least two, at least three, at least four, at least five, or at least six substitutions selected from Y37D, Y37F, Y37H, Y37L, L41F, L41H, D53A, D53G, D53R, D53H, E67A, E67T, E67G, E67K, E67R, T70A, T70K, T70E, D71S, D71A, D71Y, S72N, S72K, S72R, D73P, D73A, D73R, D73H, D73L, D73V, D76Y, D76S, D76A, N77K, N77S, N77R, M87F, M87L, M87I, Q91H, M96L, M96F, M96I, Q139L, Q139I, H145A, H145P, H145D, M149L, M149I, M149F, and R167S. In some embodiments, the modified human IL-18 polypeptide further comprises substitutions D53G, E66A, and either Q139L or Q139I, and optionally substitutions D71S and M87F.

In any of the embodiments described herein, the stabilized IL-18 polypeptide may comprise a modified IL-18 polypeptide and a fusion partner. In some embodiments, the stabilized IL-18 polypeptide comprises a modified IL-18 polypeptide and does not comprise a fusion partner.

In any of the embodiments described herein, the stabilized IL-18 polypeptide may be conjugated to a polymer, such as polyethylene glycol (PEG). Thus, in some embodiments, a conjugate is provided, comprising a stabilized IL-18 polypeptide provided herein conjugated to a polymer, such as PEG.

### Protein Variants

In certain aspects, amino acid sequence variants of the polypeptides provided herein are contemplated. For example, it may be desirable to alter the binding affinity and/or other biological properties of the polypeptide. Amino acid sequence variants of a polypeptide may be prepared by introducing appropriate modifications into the nucleotide sequence encoding the polypeptide, or by peptide synthesis. Such modifications include, for example, deletions from, and/or insertions into and/or substitutions of residues within the amino acid sequences of the polypeptide. Any combination of deletion, insertion, and substitution can be made to arrive at the final construct, provided that the final construct possesses the desired characteristics, e.g., binding.

### a) Substitution, Insertion, and Deletion Variants

In certain aspects, polypeptide variants having one or more amino acid substitutions are provided. Conservative substitutions are shown in Table 1 under the heading of “preferred substitutions”. More substantial changes are provided in Table 1 under the heading of “exemplary substitutions”, and as further described below in reference to amino acid side chain classes. Amino acid substitutions may be introduced into a polypeptide of interest and the products screened for a desired activity, e.g., increased or decreased receptor binding, increased or decreased potency, decreased immunogenicity, improved production yield, and/or improved half-life.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

A useful method for identification of residues or regions of a polypeptide that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the polypeptide with, e.g., its receptor is affected. Further substitutions may be introduced at the amino acid locations demonstrating functional sensitivity to the initial substitutions. Alternatively, or additionally, a crystal structure of a polypeptide-receptor complex may be used to identify contact points between the polypeptide and its receptor. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a polypeptide with an N-terminal methionyl residue. Other insertional variants of the polypeptide include the fusion to the N- or C-terminus, for example, to increase the serum half-life of the polypeptide.

### Exemplary Fusion Partners

In some embodiments, a stabilized IL-18 polypeptide comprises a fusion partner. In some embodiments, the fusion partner may extend the half-life of the stabilized IL-18 polypeptide and/or aid in purification of the polypeptide. In some embodiments, a fusion partner is an antigen binding domain. Various fusion partners are known in the art, including but not limited to, Fc regions, albumin, antibodies, Fabs, scFvs, and VHH domains. In some embodiments, the fusion partner is derived from a human protein. In some embodiments, the fusion partner comprises substitutions compared to the human protein from which it is derived, for example, to confer desirable properties.

### Fc Regions

In some embodiments, a stabilized IL-18 polypeptide provided herein comprises an Fc region. In various embodiments, the Fc region is a human IgG₁, IgG₂, IgG₃ or IgG₄ Fc region.

In certain aspects, one or more amino acid modifications may be introduced into the fusion partner of a stabilized IL-18 polypeptide provided herein, thereby generating a polypeptide. The polypeptide may comprise, for example, a human Fc region sequence (e.g., a human IgG₁, IgG₂, IgG₃ or IgG₄ Fc region) comprising an amino acid modification (e.g., a substitution) at one or more amino acid positions.

In certain aspects, the invention contemplates a Fc region that possesses some but not all effector functions, which make it a desirable candidate for applications in which the half-life of the polypeptide comprising the Fc region in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the polypeptide comprising the Fc region lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'tl Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the polypeptide comprising the Fc region is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Polypeptides comprising Fc regions with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain Fc regions with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, a polypeptide comprises an Fc region with one or more amino acid substitutions which improve ADCC, e.g., substitutions at positions 298, 333, and/or 334 of the Fc region (EU numbering of residues).

In certain aspects, a polypeptide comprises a Fc region with one or more amino acid substitutions which diminish FcγR binding, e.g., substitutions at positions 234 and 235 of the Fc region (EU numbering of residues). In some aspects, the substitutions are L234A and L235A (LALA). In certain aspects, the Fc region further comprises D265A and/or P329G in an Fc region derived from a human IgG₁ Fc region. In some aspects, the substitutions are L234A, L235A and P329G (LALA-PG) in an Fc region derived from a human IgG₁ Fc region. (See, e.g., WO 2012/130831). In some aspects, the substitutions are L234A, L235A and D265A (LALA-DA) in an Fc region derived from a human IgG₁ Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Polypeptides with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those polypeptides comprise a fusion partner Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues 1253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, a polypeptide comprises an Fc region with one or more amino acid substitutions, which reduce FcRn binding, e.g., substitutions at positions 253, and/or 310, and/or 435 of the Fc-region (EU numbering of residues). In certain aspects, the polypeptide comprises an Fc region with the amino acid substitutions at positions 253, 310 and 435. In some aspects, the substitutions are I253A, H310A and H435A in an Fc region derived from a human IgG1 Fc-region. See, e.g., Grevys, A., et al., J. Immunol. 194 (2015) 5497-5508.

In certain aspects, a polypeptide comprises an Fc region with one or more amino acid substitutions, which reduce FcRn binding, e.g., substitutions at positions 310, and/or 433, and/or 436 of the Fc region (EU numbering of residues). In certain aspects, the polypeptide comprises an Fc region with the amino acid substitutions at positions 310, 433 and 436. In some aspects, the substitutions are H310A, H433A and Y436A in an Fc region derived from a human IgG1 Fc-region. (See, e.g., WO 2014/177460 A1).

In certain aspects, a polypeptide comprises an Fc region with one or more amino acid substitutions which increase FcRn binding, e.g., substitutions at positions 252, and/or 254, and/or 256 of the Fc region (EU numbering of residues). In certain aspects, the polypeptide comprises an Fc region with amino acid substitutions at positions 252, 254, and 256. In some aspects, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

### B. Recombinant Methods and Compositions

In some aspects, isolated nucleic acids encoding stabilized IL-18 polypeptides as used in the methods as reported herein are provided.

In some aspects, a method of making a stabilized IL-18 polypeptide is provided, wherein the method comprises culturing a host cell comprising nucleic acid(s) encoding the polypeptide, as provided above, under conditions suitable for expression of the polypeptide, and optionally recovering the polypeptide from the host cell (or host cell culture medium).

For recombinant production of an IL-18 polypeptide, nucleic acids encoding the polypeptide, e.g., as described above, are isolated and inserted into one or more vectors for further cloning and/or expression in a host cell. Such nucleic acids may be readily isolated and sequenced using conventional procedures or produced by recombinant methods or obtained by chemical synthesis.

Suitable host cells for cloning or expression of polypeptide-encoding vectors include prokaryotic or eukaryotic cells described herein.

In some embodiments, vertebrate cells may be used as hosts. For example, mammalian cell lines that are adapted to grow in suspension may be useful. Examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0.

In some aspects, the host cell is eukaryotic, e.g., a Chinese Hamster Ovary (CHO) cell or lymphoid cell (e.g., Y0, NS0, Sp20 cell).

### C. Assays

Stabilized IL-18 polypeptides provided herein may be identified, screened for, or characterized for their physical/chemical properties and/or biological activities by various assays known in the art.

### 1. Binding Assays and Other Assays

In some aspects, a stabilized IL-18 polypeptide of the invention is tested for its binding activity, e.g., by known methods such as ELISA, Western blot, etc.

In some aspects, a stabilized IL-18 polypeptide of the invention is tested for its binding affinity for the IL-18Rα or IL-18BP, i.e., K_{D}. In some aspects, K_{D} is measured using a BIACORE® surface plasmon resonance assay. For example, an assay using a BIACORE®-2000, BIACORE®-3000 (BIAcore, Inc., Piscataway, NJ), or BIACORE® 8K is performed at 25° C. or 37° C. with immobilized target (such as biotinylated IL-18Rα or IL-18BP) on, for example, a streptavidin chip chips at ≤50 response units (RU). In another aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Target is diluted with 10 mM sodium acetate, pH 4.8, to 5 μg/ml (˜0.2 μM) before injection at a flow rate of 5 l/minute to achieve ≤50 response units (RU) of coupled protein. Following the injection of target, 1 M ethanolamine is injected to block unreacted groups. For kinetics measurements, two- or three-fold serial dilutions of stabilized IL-18 polypeptide are injected in HBS-P+ buffer (10 mM HEPES pH 7.4, 150 mM NaCl, 0.005% surfactant P20). Association rates (kₒₙ) and dissociation rates (k_{off}) are calculated using a simple one-to-one (1:1) Langmuir binding model (BIACORE ©Evaluation Software version 3.2 or BIACORE® 8K evaluation software) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999).

In another exemplary assay using a BIAcore™ T200 machine, for example, stabilized IL-18 polypeptides comprising human IgG1 constant regions are captured on a protein A chip to achieve approximately 300 RU. In some such embodiments, serial dilutions of purified target are injected in HBS-P buffer with additional 3 mM CaCl₂) at 37° C. with a flow rate of 100 μL/min. Association rates (ka) and dissociation rates (kd) are calculated using a 1:1 Langmuir binding model (BIAcore™ T200 Evaluation Software version 2.0, for example). The equilibrium dissociation constant (K_{D}) may be calculated as the ratio kd/ka.

If the on-rate exceeds 10⁶ M⁻¹ s⁻¹ by a surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM stabilized IL-18 polypeptide in PBS, pH 7.2, in the presence of increasing concentrations of target as measured in a spectrometer, such as a stop-flow equipped spectrophometer (Aviv Instruments) or a 8000-series SLM-AMINCO™ spectrophotometer (ThermoSpectronic) with a stirred cuvette.

In some embodiments, a reporter assay is used to determine potency of a stabilized IL-18 polypeptide, for example, as described herein. An exemplary assay is as follows. HEK293 cells are stably transfected to express IL-18 receptor. IL-18 receptor signaling drives expression of secreted alkaline phosphatase (SEAP), which is then measured using a colorimetric reagent.

In a further exemplary assay, an IL-18 polypeptide may be tested for potency using a native T-cell assay by measuring IFNγ production, for example, as described herein. An exemplary assay is as follows. Peripheral blood mononuclear cells are isolated from whole blood, for example, using SepMate Isolation Tubes (STEMCELL Technologies, 15460). Human T cells are isolated from PBMCs, for example, using an immunomagnetic negative selection kit (STEMCELL Technologies, 17951). Cells are seeded 1×10⁴ cells/well on a 384 well-plate pre-coated with CD3 and CD28. Cells are stimulated with a range of concentrations of the stabilized IL-18 polypeptides, for example, from 0.5 to 10,000 pM, or from 5 to 100,000 pM. Cells are incubated at 37° C. and 5% CO2 in 10% FBS RPMI media supplemented with NEAA, sodium pyruvate, β-mercaptoethanol, and human IL-12. After 24 h, IFN-γ production in supernatant is measured, for example, using human IFN-γ HTRF kit (Cisbio, 62HIFNGPEG).

In certain embodiments, a stabilized IL-18 protein is tested for stimulation of peripheral blood mononuclear cells are isolated from whole blood via SepMate Isolation Tubes (STEMCELL Technologies, 15460). Human T cells are isolated from PBMCs by a immunomagnetic negative selection kit (STEMCELL Technologies, 17951). Cells are seeded 1×104 cells/well on a 384 well-plate pre-coated with CD3 (5 ug/mL, Thermofisher, 16-0037-85) and CD28 (5 ug/mL, Biosciences 555725). For the human IL-18 stimulation assay, cells have concentrations ranging from 0.5 to 10,000 pM. For more attenuated IL-18 molecules, concentrations are increased ranging from 5 to 100,000 pM. All treatments are incubated at 37° C. and 5% C02 in 10% FBS RPMI media supplemented with NEAA (dilute 1:100, Gibco 11140-050), Sodium Pyruvate (dilute 1:100, Gibco 11360-070), b-Mercaptoethanol (dilute 1:1000, Gibco 21985-023,) and human IL-12 (10 ng/mL, R&D, 219-IL-025/CF). After 24 h, IFN-γ production in supernatant is measured using human IFN-γ HTRF kit (Cisbio, 62HIFNGPEG).

### D. Methods and Compositions for Diagnostics and Detection

In certain aspects, any of the stabilized IL-18 polypeptides provided herein is useful for detecting the presence of IL-18BP or IL-18Rα in a biological sample. The term “detecting” as used herein encompasses quantitative or qualitative detection. In certain aspects, a biological sample comprises a biological fluid, cell, or tissue, such as sputum, secretory cells, airway epithelial cells, immune cells, lung cells or tissue, or bronchial cells or tissue.

In some aspects, a stabilized IL-18 polypeptide for use in a method of diagnosis or detection is provided. In a further aspect, a method of detecting the presence of IL-18BP or IL-18Rα in a biological sample is provided. In certain aspects, the method comprises contacting the biological sample with a stabilized IL-18 polypeptide as described herein under conditions permissive for binding of the stabilized IL-18 polypeptide to IL-18BP or IL-18Rα, and detecting whether a complex is formed between the stabilized IL-18 polypeptide and IL-18BP or IL-18Rα. Such method may be an in vitro or in vivo method. In some aspects, a stabilized IL-18 polypeptide is used to select subjects eligible for therapy with an IL-18BP or IL-18Rα, e.g., where IL-18BP or IL-18Rα is a biomarker for selection of patients.

In certain aspects, labeled stabilized IL-18 polypeptide are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### E. Pharmaceutical Compositions

In a further aspect, provided are pharmaceutical compositions comprising any of the polypeptides provided herein, e.g., for use in any of the below therapeutic methods. In some aspects, a pharmaceutical composition comprises any of the polypeptides provided herein and a pharmaceutically acceptable carrier. In some aspects, a pharmaceutical composition comprises any of the polypeptides provided herein and at least one additional therapeutic agent, e.g., as described below.

Pharmaceutical compositions of stabilized IL-18 protein as described herein are prepared by mixing such polypeptide having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In some aspects, a sHASEGP is combined with one or more additional glycosaminoglycanases such as chondroitinases.

The pharmaceutical composition herein may also contain more than one active ingredient as necessary for the particular indication being treated, preferably those with complementary activities that do not adversely affect each other. For example, it may be desirable to further provide a chemotherapeutic agent and/or immunooncology agent. In some embodiments, an additional therapeutic agent is an immunooncology agent. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the polypeptide, which matrices are in the form of shaped articles, e.g., films, or microcapsules.

The pharmaceutical compositions to be used for in vivo administration are generally sterile. Sterility may be readily accomplished, e.g., by filtration through sterile filtration membranes.

### F. Therapeutic Methods and Routes of Administration

Any of the stabilized IL-18 polypeptides provided herein may be used in therapeutic methods.

In one aspect, a stabilized IL-18 polypeptide for use as a medicament is provided. In further aspects, a stabilized IL-18 polypeptide for use in treating cancer is provided. Examples of cancer include, but are not limited to, carcinoma, adenocarcinoma, lymphoma (e.g., Hodgkin's and non-Hodgkin's lymphoma), blastoma, sarcoma, and leukemia. In certain embodiments, cancers that are amenable to treatment using the polypeptides of the invention include adenocarcinomas (e.g., colorectal adenocarcinoma, gastric adenocarcinoma, or pancreatic adenocarcinoma), which may be metastatic adenocarcinomas (e.g., metastatic colorectal adenocarcinoma, metastatic gastric adenocarcinoma, or metastatic pancreatic adenocarcinoma), esophageal cancer, gastric or stomach cancer, small intestine cancer, large intestine cancer, small cell lung cancer, glioblastoma, neuroblastoma, melanoma, breast carcinoma, gastric cancer, colorectal cancer (CRC), hepatocellular carcinoma, breast cancer, rectal cancer, non-small cell lung cancer, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, and multiple myeloma. In other embodiments, the cancer is selected from a class of mature B-Cell cancers excluding Hodgkin's Lymphoma but including germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma. In some embodiments, a stabilized IL-18 polypeptide is for use in treating an infectious disease.

In certain aspects, a stabilized IL-18 polypeptide for use in a method of treatment is provided. In certain aspects, the invention provides a stabilized IL-18 polypeptide for use in a method of treating an individual having cancer comprising administering to the individual an effective amount of the stabilized IL-18 polypeptide. In one such aspect, the method further comprises administering to the individual an effective amount of at least one additional therapeutic agent (e.g., one, two, three, four, five, or six additional therapeutic agents), e.g., as described below. In further aspects, the invention provides a stabilized IL-18 polypeptide for use in activating the IL-18 receptor on a cell. In certain aspects, the invention provides a stabilized IL-18 polypeptide for use in a method of activating the IL-18 receptor on a cell in an individual comprising administering to the individual an effective amount of the stabilized IL-18 polypeptide to activate the IL-18 receptor on a cell.

In further aspects, the invention provides a stabilized IL-18 polypeptide for use in inducing IFNγ expression in a lymphocyte. In certain aspects, the invention provides a stabilized IL-18 polypeptide for use in a method of inducing IFNγ expression in a lymphocyte in an individual comprising administering to the individual an effective amount of the stabilized IL-18 polypeptide to induce IFNγ expression in a lymphocyte.

In further aspects, the invention provides a stabilized IL-18 polypeptide for use in activating a lymphocyte. In certain aspects, the invention provides a stabilized IL-18 polypeptide for use in a method of activating a lymphocyte in an individual comprising administering to the individual an effective amount of the stabilized IL-18 polypeptide to activate a lymphocyte.

In a further aspect, the invention provides for the use of a stabilized IL-18 polypeptide in the manufacture or preparation of a medicament. In one aspect, the medicament is for treatment of cancer. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer. In some embodiments, a stabilized IL-18 polypeptide is for use in treating an infectious disease. In a further aspect, the medicament is for use in a method of treating cancer comprising administering to an individual having cancer an effective amount of the medicament. In one such aspect, the method further comprises administering to the individual an effective amount of at least one additional therapeutic agent, e.g., as described below.

In a further aspect, the invention provides a method for treating a cancer. In one aspect, the method comprises administering to an individual having such cancer an effective amount of a stabilized IL-18 polypeptide. In one such aspect, the method further comprises administering to the individual an effective amount of at least one additional therapeutic agent, as described below.

An “individual” according to any of the aspects provided herein is preferably a human.

In a further aspect, the invention provides pharmaceutical compositions comprising any of the stabilized IL-18 polypeptides provided herein, e.g., for use in any of the above therapeutic methods. In one aspect, a pharmaceutical composition comprises any of the stabilized IL-18 polypeptides provided herein and a pharmaceutically acceptable carrier. In another aspect, a pharmaceutical composition comprises any of the stabilized IL-18 polypeptides provided herein and at least one additional therapeutic agent, e.g., as described below.

Stabilized IL-18 polypeptides of the invention can be administered alone or used in a combination therapy. For instance, the combination therapy includes administering a stabilized IL-18 polypeptides of the invention and administering at least one additional therapeutic agent (e.g. one, two, three, four, five, or six additional therapeutic agents). In some embodiments, a stabilized IL-18 polypeptide is administered in combination with an immunooncology agent and/or a chemotherapeutic agent.

In certain aspects, the combination therapy comprises administering a stabilized IL-18 polypeptide of the invention and administering at least one additional therapeutic agent, such as an antibody that binds a tumor associated antigen; a CD28, OX40, GITR, CD137, CD27, CD40, ICOS, HVEM, NKG2D, MICA, 2B4, IL-2, IL-12, IL-15, IL-27, IFNγ, IFNα, TNFα, IL-1, CDN, HMGB1, or TLR agonist; or is a PD-1, PD-L1, CTLA-4, TIM-3, BTLA, VISTA, LAG-3, CD47, SIRPa, B7H4, CD96, TIGIT, CD226, prostaglandin, VEGF, endothelin B, IDO, arginase, MICA/MICB, TIM-3, IL-10, IL-4, or IL-13 antagonist.

In some embodiments, a stabilized IL-18 polypeptide provided herein is administered with at least one immunooncology agent. In some embodiments, the immunooncology agent is an agonist directed against an activating co-stimulatory molecule. In some embodiments, the immunooncology agent is an immune checkpoint inhibitor. In various embodiments, the immunooncology agent is an antibody.

Without intending to be bound to any particular theory, it is thought that enhancing T-cell stimulation, by promoting an activating co-stimulatory molecule or by inhibiting a negative co-stimulatory molecule, may promote tumor cell death thereby treating or delaying progression of cancer. Therefore, in some instances, a stabilized IL-18 polypeptide may be administered in combination with an agonist directed against an activating co-stimulatory molecule. In some instances, an activating co-stimulatory molecule may include CD28, OX40, GITR, CD137, CD27, CD40, ICOS, HVEM, NKG2D, MICA, 2B4, IL-2, IL-12, IL-15, IL-27, IFNγ, IFNα, TNFα, IL-1, CDN, HMGB1, or TLR. In some instances, the agonist directed against an activating co-stimulatory molecule is an agonist antibody that binds to CD28, OX40, GITR, CD137, CD27, CD40, ICOS, HVEM, NKG2D, MICA, 2B4, IL-2, IL-12, IL-15, IL-27, IFNγ, IFNα, TNFα, IL-1, CDN, HMGB1, or TLR. In some instances, a stabilized IL-18 polypeptide may be administered in combination with an antagonist directed against an inhibitory co-stimulatory molecule. In some instances, an inhibitory co-stimulatory molecule may include PD-1, PD-L1, CTLA-4, TIM-3, BTLA, VISTA, LAG-3, CD47, SIRPα, B7H4, CD96, TIGIT, CD226, prostaglandin, VEGF, endothelin B, IDO, arginase, MICA/MICB, TIM-3, IL-10, IL-4, or IL-13. In some instances, the antagonist directed against an inhibitory co-stimulatory molecule is an antagonist antibody that binds to PD-1, PD-L1, CTLA-4, TIM-3, BTLA, VISTA, LAG-3, CD47, SIRPα, B7H4, CD96, TIGIT, CD226, prostaglandin, VEGF, endothelin B, IDO, arginase, MICA/MICB, TIM-3, IL-10, IL-4, or IL-13.

In some instances, a stabilized IL-18 polypeptide may be administered in combination with an antagonist directed against CTLA-4 (also known as CD152), e.g., a blocking antibody. In some instances, a stabilized IL-18 polypeptide may be administered in combination with ipilimumab. In some instances, a stabilized IL-18 polypeptide may be administered in combination with tremelimumab (also known as ticilimumab).

In some instances, a stabilized IL-18 polypeptide may be administered in combination with an antagonist directed against B7-H3 (also known as CD276), e.g., a blocking antibody. In some instances, a stabilized IL-18 polypeptide may be administered in combination with MGA271.

In some instances, a stabilized IL-18 polypeptide may be administered in combination with an antagonist directed against a TGF-beta, e.g., metelimumab, fresolimumab, or LY2157299.

In some instances, a stabilized IL-18 polypeptide may be administered in combination with a treatment including adoptive transfer of a T cell (e.g., a cytotoxic T cell or cytotoxic lymphocyte (CTL)) expressing a chimeric antigen receptor (CAR). In some instances, a stabilized IL-18 polypeptide may be administered in combination with a treatment including adoptive transfer of a T cell including a dominant-negative TGF-beta receptor, e.g., a dominant-negative TGF-beta type II receptor.

In some instances, a stabilized IL-18 polypeptide may be administered in combination with an agonist directed against CD137 (also known as TNFRSF9, 4-1BB, or ILA), e.g., an activating antibody. In some instances, a stabilized IL-18 polypeptide may be administered in combination with urelumab. In some instances, a stabilized IL-18 polypeptide may be administered in combination with utomilumab. In some instances, a stabilized IL-18 polypeptide may be administered in combination with INBRX-105.

In some instances, a stabilized IL-18 polypeptide may be administered in combination with an agonist directed against CD40, e.g., an activating antibody. In some instances, a stabilized IL-18 polypeptide may be administered in combination with CP-870893.

In some instances, a stabilized IL-18 polypeptide may be administered in combination with APX005M. In some instances, a stabilized IL-18 polypeptide may be administered in combination with an agonist directed against OX40 (also known as CD134), e.g., an activating antibody. In some instances, a stabilized IL-18 polypeptide may be administered in combination with an anti-OX40 antibody (e.g., AgonOX). In some instances, a stabilized IL-18 polypeptide may be administered in combination with PF-04518600 (PF-8600). In some instances, a stabilized IL-18 polypeptide may be administered in combination with MEDI0562, MEDI6469, and/or MEDI6383. In some instances, a stabilized IL-18 polypeptide may be administered in combination with GSK3174998. In some instances, a stabilized IL-18 polypeptide may be administered in combination with BMS986178.

In some instances, a stabilized IL-18 polypeptide may be administered in combination with an agonist directed against CD27, e.g., an activating antibody. In some instances, a stabilized IL-18 polypeptide may be administered in combination with varlilumab.

In some instances, a stabilized IL-18 polypeptide may be administered in combination with an agonist directed against ICOS. In some instances, a stabilized IL-18 polypeptide may be administered in combination with vopratelimab. In some instances, a stabilized IL-18 polypeptide may be administered in combination with GSK3359609.

In some instances, a stabilized IL-18 polypeptide may be administered in combination with an IL-15 agonist.

In some instances, a stabilized IL-18 polypeptide may be administered in combination with an IL-27 agonist.

In some instances, a stabilized IL-18 polypeptide may be administered in combination with an agonist directed against GITR. In some instances, a stabilized IL-18 polypeptide may be administered in combination with TRX 518-001. In some instances, a stabilized IL-18 polypeptide may be administered in combination with MK-4166. In some instances, a stabilized IL-18 polypeptide may be administered in combination with BMS-986156. In some instances, a stabilized IL-18 polypeptide may be administered in combination with INCAGN01876.

In some instances, a stabilized IL-18 polypeptide may be administered in combination with an agonist directed against CD70. In some instances, a stabilized IL-18 polypeptide may be administered in combination with cusatuzumab.

In some instances, a stabilized IL-18 polypeptide may be administered in combination with an antagonist directed against VISTA. In some instances, the VISTA antagonist is CA-170.

In some instances, a stabilized IL-18 polypeptide may be administered in combination with an antagonist directed against CCR4. In some instances, the CCR4 antagonist is mogamulizumab.

In some instances, a stabilized IL-18 polypeptide may be administered in combination with an antagonist directed against B7-H3. In some instances, the B7-H3 antagonist is MGD009. In some instances, the B7-H3 antagonist is 8H9.

In some instances, a stabilized IL-18 polypeptide may be administered in combination with an antagonist directed against TIM-3. In some instances, the TIM-3 antagonist is TSR-022. In some instances, the TIM-3 antagonist is MBG453. In some instances, the TIM-3 antagonist is Sym023. In some instances, the TIM-3 antagonist is oleclumab.

In some instances, a stabilized IL-18 polypeptide may be administered in combination with an antagonist directed against LAG-3. In some instances, the LAG-3 antagonist is relatlimab. In some instances, the LAG-3 antagonist is IMP321 (eftilagimod alpha).

In some instances, the LAG-3 antagonist is LAG525.

In some instances, a stabilized IL-18 polypeptide may be administered in combination with an antagonist directed against KIR (2DL1-3). In some instances, the KIR antagonist is lirilumab.

In some instances, a stabilized IL-18 polypeptide may be administered in combination with an antagonist directed against IDO-1,2. In some instances, the IDO-1,2 antagonist is indoximod. In some instances, the IDO-1,2 antagonist is epacadostat. In some instances, a stabilized IL-18 polypeptide may be administered in combination with an antagonist directed against indoleamine-2,3-dioxygenase (IDO). In some instances, the IDO antagonist is 1-methyl-D-tryptophan (also known as 1-D-MT).

In some instances, a stabilized IL-18 polypeptide may be administered in combination with an antagonist directed against TIGIT. In some instances, the TIGIT antagonist is tislelizumab. In some instances, the TIGIT antagonist is tiragolumab. In some instances, the TIGIT antagonist is BMS-986207. In some instances, the TIGIT antagonist is MTIG7192A. In some instances, the TIGIT antagonist is AB154.

In some instances, a stabilized IL-18 polypeptide may be administered in combination with an antagonist directed against A2aR. In some instances, the A2aR antagonist is Ciforadenant.

In some instances, a stabilized IL-18 polypeptide may be administered in combination with an antagonist directed against transforming growth factor 3. In some instances, the transforming growth factor R antagonist is M7824. In some instances, the transforming growth factor β antagonist is calunisertib.

In some instances, a stabilized IL-18 polypeptide may be administered in combination with an antagonist directed against CD47. In some instances, the CD47 antagonist is TTI-621. In some instances, the CD47 antagonist is ALX148 (evorpacept). In some instances, the CD47 antagonist is magrolimab.

In some instances, a stabilized IL-18 polypeptide may be administered in combination with an antagonist directed against CD73. In some instances, the CD73 is oleclumab.

In some instances, a stabilized IL-18 polypeptide may be administered in combination with an agent directed to toll-like receptors. In some instances, a stabilized IL-18 polypeptide may be administered in combination with PolyICIC. In some instances, a stabilized IL-18 polypeptide may be administered in combination with leftitolimod. In some instances, a stabilized IL-18 polypeptide may be administered in combination with SD101. In some instances, a stabilized IL-18 polypeptide may be administered in combination with DSP-0509.

In some instances, a stabilized IL-18 polypeptide may be administered in combination with Rintatolimod. In some instances, a stabilized IL-18 polypeptide may be administered in combination with CMP-001.

In some instances, a stabilized IL-18 polypeptide may be administered in combination with an agent directed to the interleukin 2 receptor. In some instances, a stabilized IL-18 polypeptide may be administered in combination with NKTR-214. In some instances, a stabilized IL-18 polypeptide may be administered in combination with R06874281. In some instances, a stabilized IL-18 polypeptide may be administered in combination with THOR-707.

In some instances, a stabilized IL-18 polypeptide may be administered in combination with an arginase inhibitor. In some instances, a stabilized IL-18 polypeptide may be administered in combination with CB-1158.

In some instances, a stabilized IL-18 polypeptide may be administered in combination with an oncolytic peptide. In some instances, a stabilized IL-18 polypeptide may be administered in combination with LTX-315.

In some instances, a stabilized IL-18 polypeptide may be administered in combination with interleukin 10. In some instances, a stabilized IL-18 polypeptide may be administered in combination with pegilodecakin.

In another embodiment, provided are methods of using stabilized IL-18 polypeptides to treat and/or delay progression of cancer in combination with a PD-1 axis binding antagonist. Further provided herein are methods of enhancing immune function in an individual having cancer comprising administering to the individual an effective amount of a stabilized IL-18 polypeptide and an effective amount of a PD-1 axis binding antagonist. A PD-1 axis binding antagonist includes a PD-1 binding antagonist, a PD-L1 binding antagonist and a PD-L2 binding antagonist.

The term “PD-1 axis binding antagonist” is a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonists” is a molecule that decreases, blocks, inhibits, abrogates or interferes with signal transduction resulting from the interaction of PD-1 with one or more of its binding partners, such as PDL1, PDL2. In some embodiments, the PD-1 binding antagonist is a molecule that inhibits the binding of PD-1 to its binding partners. In a specific aspect, the PD-1 binding antagonist inhibits the binding of PD-1 to PDL1 and/or PDL2.

For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PDL1 and/or PDL2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is nivolumab. In another specific aspect, a PD-1 binding antagonist is pembrolizumab. In another specific aspect, a PD-1 binding antagonist is CT-011 (also known as hBAT or hBAT-1). In yet another specific aspect, a PD-1 binding antagonist is AMP-224 (also known as B7-DCIg).

The term “PDL1 binding antagonists” is a molecule that decreases, blocks, inhibits, abrogates or interferes with signal transduction resulting from the interaction of PDL1 with either one or more of its binding partners, such as PD-1, B7-1. In some embodiments, a PDL1 binding antagonist is a molecule that inhibits the binding of PDL1 to its binding partners. In a specific aspect, the PDL1 binding antagonist inhibits binding of PDL1 to PD-1 and/or B7-1. In some embodiments, the PDL1 binding antagonists include anti-PDL1 antibodies, antigen binding fragments thereof, immunoadhesins, fusion proteins, oligopeptides and other molecules that decrease, block, inhibit, abrogate or interfere with signal transduction resulting from the interaction of PDL1 with one or more of its binding partners, such as PD-1, B7-1. In one embodiment, a PDL1 binding antagonist reduces the negative co-stimulatory signal mediated by or through cell surface proteins expressed on T lymphocytes mediated signaling through PDL1 so as to render a dysfunctional T-cell less dysfunctional (e.g., enhancing effector responses to antigen recognition). In some embodiments, a PDL1 binding antagonist is an anti-PDL1 antibody. In a specific aspect, an anti-PDL1 antibody is atezolizumab. In some embodiments, an anti-PDL1 antibody is avelumab. In some embodiments, an anti-PDL1 antibody is durvalumab.

In some aspects, the stabilized IL-18 polypeptides are for use in a combination therapy for the treatment of cancer. In one embodiment, the combination therapy comprises administering a stabilized IL-18 polypeptide and administering at least one checkpoint inhibitor, such as an anti-PD-1 antibody, including pembrolizumab and nivolumab, or an anti-CTLA-4 antibody, including ipilimumab. In one embodiment, the combination therapy comprises administering a stabilized IL-18 polypeptide and administering an anti-CTLA-4 antibody and an anti-PD-1 antibody. In one embodiment, the combination therapy comprises administering an stabilized IL-18 polypeptide and administering ipilimumab and nivolumab.

In some aspects, the stabilized IL-18 polypeptide are for use in a combination therapy for the treatment melanomas having BRAF V600 mutations. In one embodiment, the combination therapy comprises administering a stabilized IL-18 polypeptide and administering at least one MAPK pathway inhibitor, such as murafenib, cobimetinib; dabrafenib, or trametinib. In one embodiment, the combination therapy comprises administering a stabilized IL-18 polypeptide and administering at least one BRAF inhibitor, such as vemurafenib, cobimetinib, or dabrafenib, and at least one MEK inhibitor, such as trametinib. In some embodiments, a combination therapy comprises administering a stabilized IL-18 polypeptide and vemurafenib plus cobimetinib; or dabrafenib plus trametinib. In some embodiments, a combination therapy comprises administering a stabilized IL-18 polypeptide, pembrolizumab or nivolumab, and a BRAF inhibitor, such as vemurafenib, cobimetinib, or dabrafenib.

Such combination therapies noted above encompass combined administration (where two or more therapeutic agents are included in the same or separate pharmaceutical compositions), and separate administration, in which case, administration of the stabilized IL-18 polypeptide of the invention can occur prior to, simultaneously, and/or following, administration of the additional therapeutic agent or agents. In one aspect, administration of the stabilized IL-18 polypeptide and administration of an additional therapeutic agent occur within about one month, or within about one, two or three weeks, or within about one, two, three, four, five, or six days, of each other. In one aspect, the stabilized IL-18 polypeptide and additional therapeutic agent are administered to the patient on Day 1 of the treatment. Stabilized IL-18 polypeptides of the invention can also be used in combination with surgery, chemotherapy (i.e., in combination with a chemotherapeutic agent), and/or radiation therapy.

A stabilized IL-18 polypeptide of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Stabilized IL-18 polypeptides of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The stabilized IL-18 polypeptide need not be, but is optionally formulated with one or more agents currently used to prevent or treat the disorder in question. The effective amount of such other agents depends on the amount of the stabilized IL-18 polypeptide present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a stabilized IL-18 polypeptide of the invention (when used alone or in combination with one or more other additional therapeutic agents) will depend on the type of disease to be treated, the type of stabilized IL-18 polypeptide, the severity and course of the disease, whether the stabilized IL-18 polypeptide is administered for preventive or therapeutic purposes, previous therapy, the patient's clinical history and response to the stabilized IL-18 polypeptide, and the discretion of the attending physician. The stabilized IL-18 polypeptide is suitably administered to the patient at one time or over a series of treatments. For repeated administrations over several days or longer, depending on the condition, the treatment would generally be sustained until a desired suppression of disease symptoms occurs. Such doses may be administered intermittently, e.g., every week or every three weeks (e.g., such that the patient receives from about two to about twenty, or, e.g., about six doses of the stabilized IL-18 polypeptide). An initial higher loading dose, followed by one or more lower doses may be administered. The progress of this therapy can be monitored by conventional techniques and assays.

### G. Articles of Manufacture

In some aspects of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a polypeptide of the invention. The label or package insert indicates that the composition is used for treating the condition of choice. Moreover, the article of manufacture may comprise (a) a first container with a composition contained therein, wherein the composition comprises a polypeptide of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### III. EXAMPLES

The following are examples of methods and compositions of the invention. It is understood that various other embodiments may be practiced, given the general description provided above.

### Example 1: Materials & Methods

### Recombinant Proteins

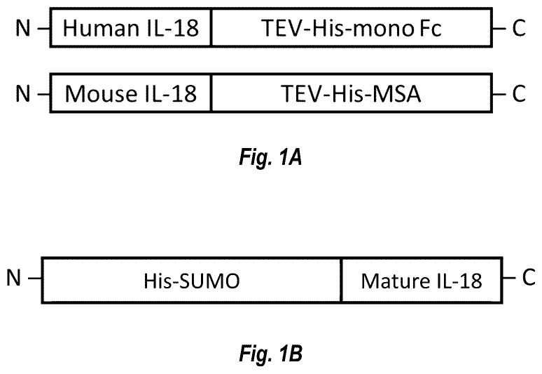

Mature human IL-18 variants were produced in mammalian cells (HEK293 or CHO) as recombinant fusions with an ectopic signal peptide at the N-terminus for secretion, followed by mature IL18 (Y37-D193), TEV protease cleavage sequence, His tag and monomeric human Fc (FIG. 1A). Mature mouse IL-18 variants were expressed in mammalian cells (HEK293 or CHO) as recombinant fusions with an ectopic signal peptide at the N-terminus for secretion, followed by mature IL18 (N36-S192), TEV protease cleavage sequence, His tag and mouse serum albumin (MSA) (FIG. 1A). The proteins were purified from the conditioned media via affinity chromatography, followed by size exclusion chromatography (SEC). The C-terminal tags were removed by TEV protease digestion, and the protease and digested C-terminal tags were separated from IL-18 by subtractive affinity chromatography.

Mature human and mouse wild-type IL-18 were produced in E. coli as recombinant fusions with an N-terminal His/SUMO tag. Following cell lysis and clarification, the protein was purified via affinity chromatography followed by SEC. The N-terminal His/SUMO tags were removed by cleavage with SUMO protease ULP1 and the digestion products were separated from IL-18 by subtractive affinity chromatography.

For determination of binding kinetics constants, recombinant human and mouse IL-18Rα, human IL-18BP (isoform A), and mouse IL-18BP (isoform D) extracellular domains were produced in mammalian cells, biotinylated, and purified by affinity chromatography, followed by SEC.

### Thermostability of IL-18

For thermostability determinations of WT IL-18 and the IL-18 variants, proteins were formulated at 0.5 mg/ml in 20 mM HEPES pH 7.2, 150 mM NaCl (non-reduced) or 20 mM HEPES pH 7.2, 150 mM NaCl, 10 mM TCEP (reduced). Differential scanning fluorimetry (DSF) was performed to understand effects of the amino acid changes on thermostability of the proteins relative to WT. DSF monitors thermal unfolding of proteins in the presence of a fluorescent dye and is typically performed by using a real-time PCR instrument. SYPRO orange dye (Invitrogen, catalog #S6650) is diluted 1:20 in 20 mM HEPES pH 7.2, 150 mM NaCl. One μl of diluted dye is added to 24 μl IL-18 protein in a well. As the temperature increases from 20° C. to 100° C. in a real-time PCR instrument (Bio-Rad CFX 96 RT), the fluorescence intensity is plotted and the inflection point of the transition curve (Tm) is calculated using, for example, the Boltzmann equation. See Nature Protocols, 2007, 2:2212-2221.

### Surface Plasmon Resonance

SPR experiments were performed using a Biacore 8K (Cytiva) at a temperature of 37° C. Biotinylated human or mouse IL-18Rα and IL-18BP were immobilized onto Biacore streptavidin sensor chip (Series S Sensor Chip SA, Cytiva) to yield an Rmax ≤50 RU. Measurements were made with five, three-fold dilutions into HBS-P+ buffer (10 mM HEPES pH 7.4, 150 mM NaCl, 0.005% surfactant P20). Interactions were evaluated using the single-cycle kinetics method and analyzed with the Biacore 8K evaluation software and fit to a 1:1 binding model.

### Potency Assay (T-Cell Assay)

Peripheral blood mononuclear cells were isolated from whole blood via SepMate Isolation Tubes (STEMCELL Technologies, 15460). Human T cells were isolated from PBMCs by a immunomagnetic negative selection kit (STEMCELL Technologies, 17951). Cells were seeded 1×10⁴ cells/well on a 384 well-plate pre-coated with CD3 (5 ug/mL, Thermofisher, 16-0037-85) and CD28 (5 ug/mL, Biosciences 555725). For the human IL-18 stimulation assay cells were stimulated with concentrations ranging from 0.5 to 10,000 pM. For more attenuated IL-18 molecules, concentrations were increased ranging from 5 to 100,000 pM. All treatments were incubated at 37° C. and 5% C02 in 10% FBS RPMI media supplemented with NEAA (dilute 1:100, Gibco 11140-050), sodium pyruvate (dilute 1:100, Gibco 11360-070), β-mercaptoethanol (dilute 1:1000, Gibco 21985-023), and human IL-12 (10 ng/mL, R&D, 219-IL-025/CF). After 24 h, IFN-γ production in supernatant was measured using human IFN-γ HTRF kit (Cisbio, 62HIFNGPEG).

### Example 2: Wild-Type IL-18 is Labile and Challenging to Produce Recombinantly in Mammalian Cells

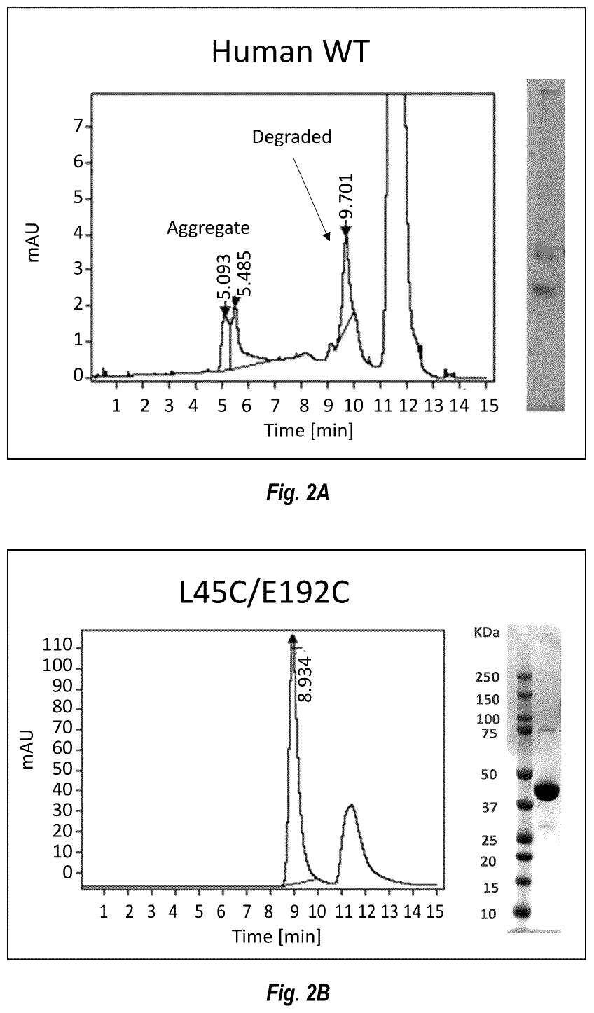

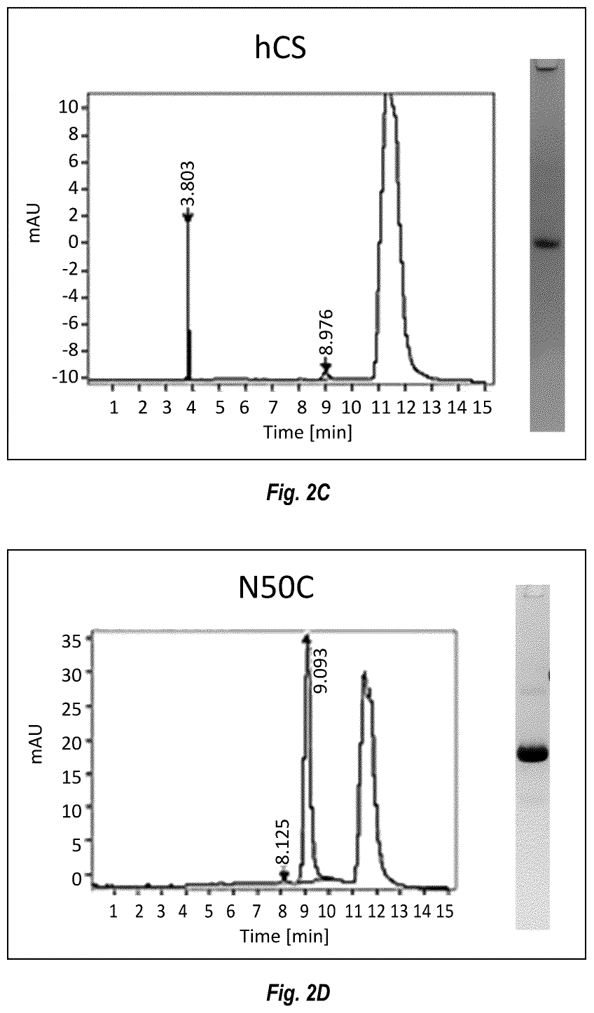

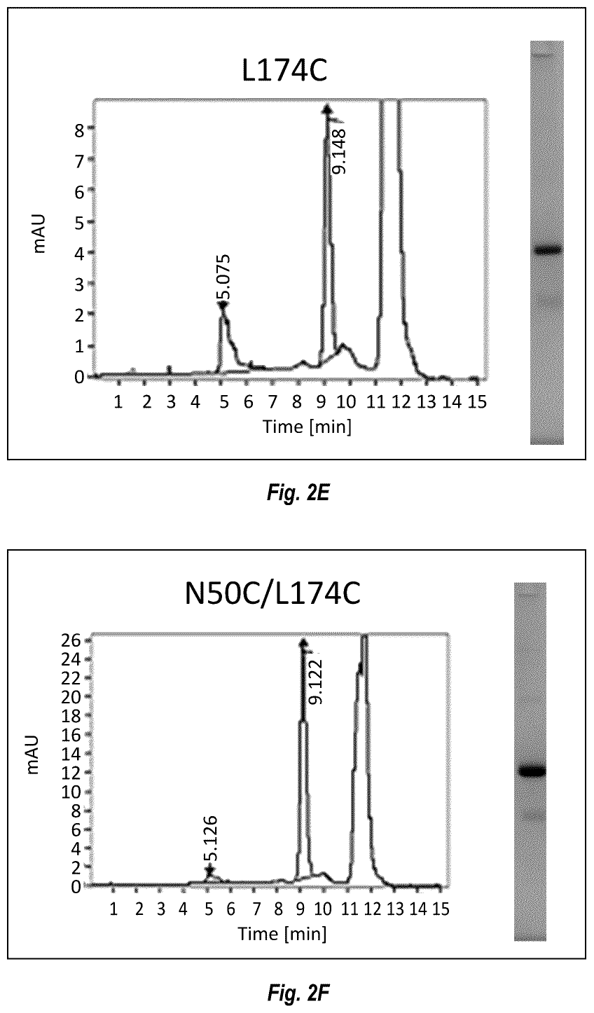

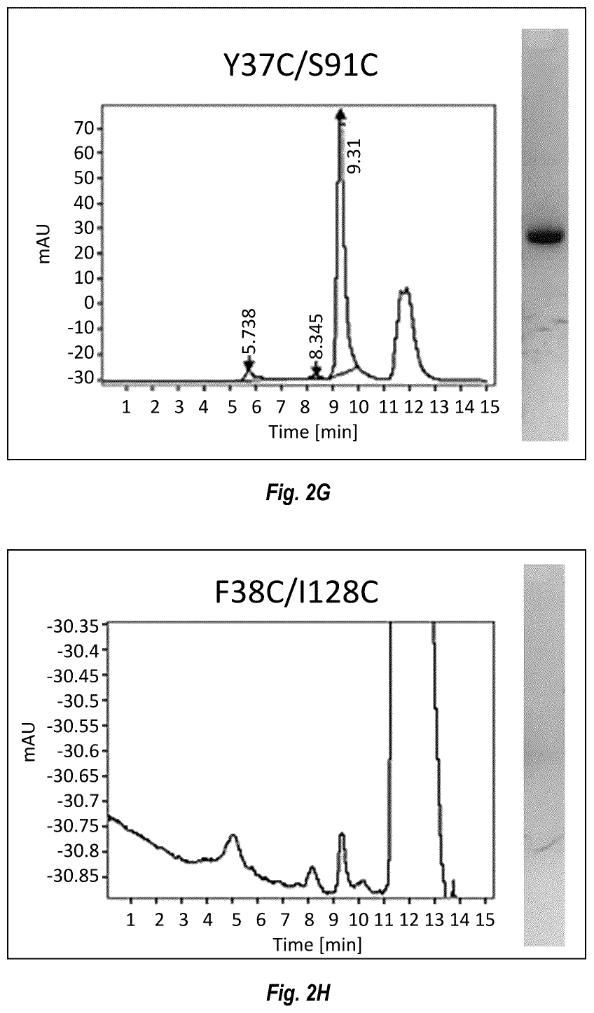

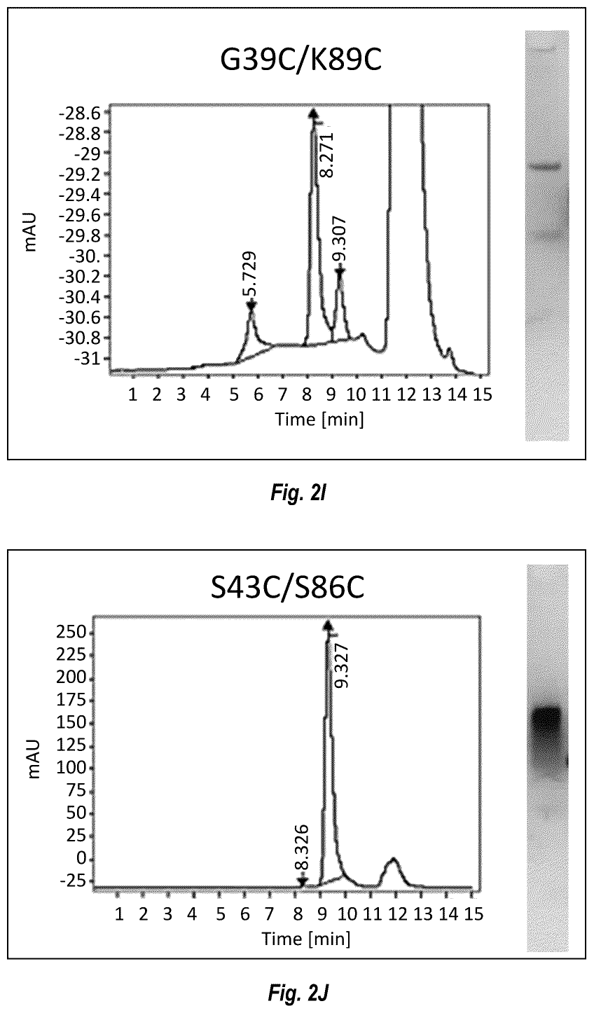

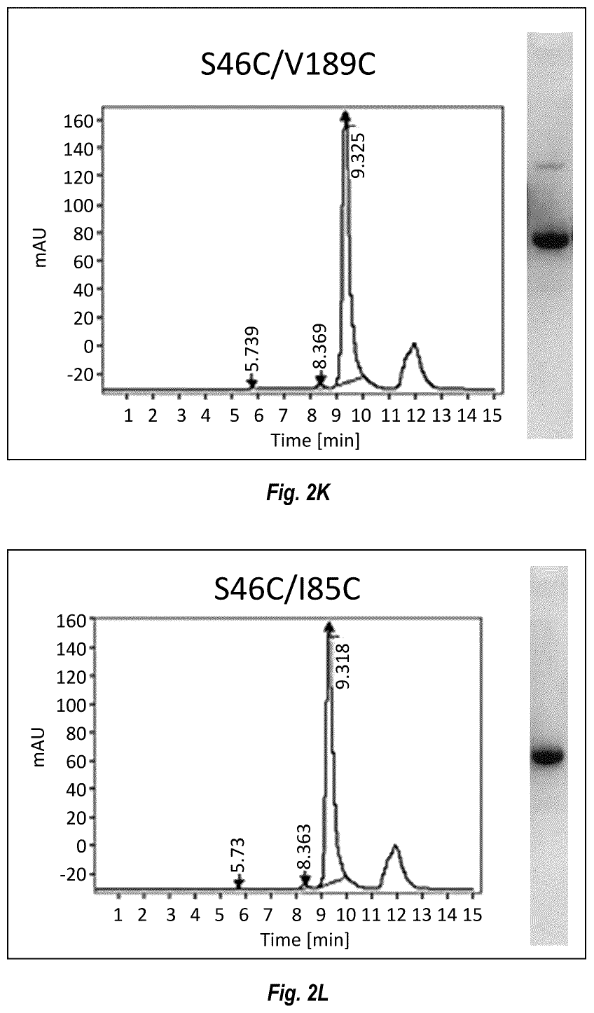

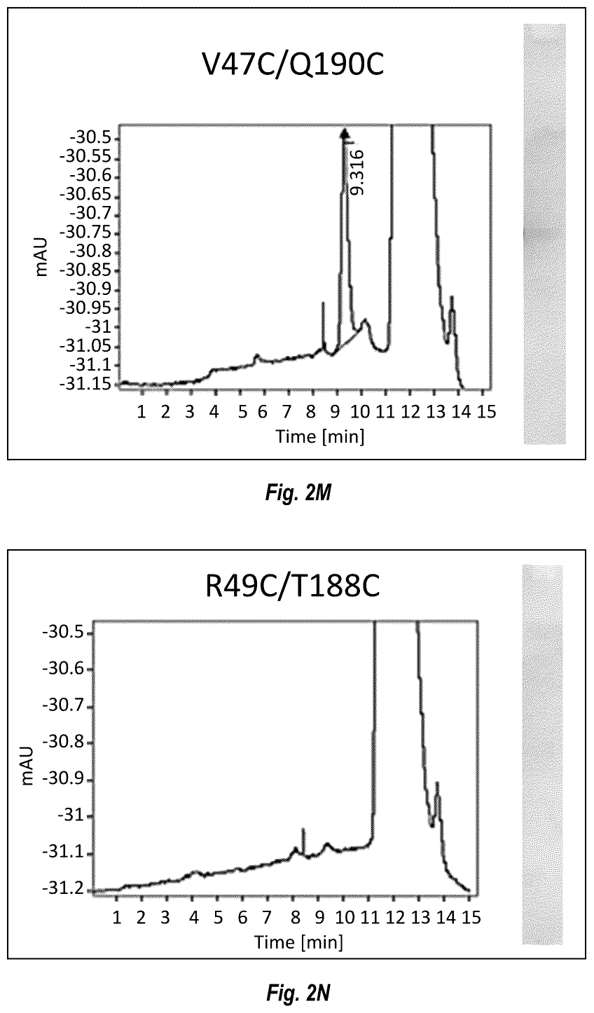

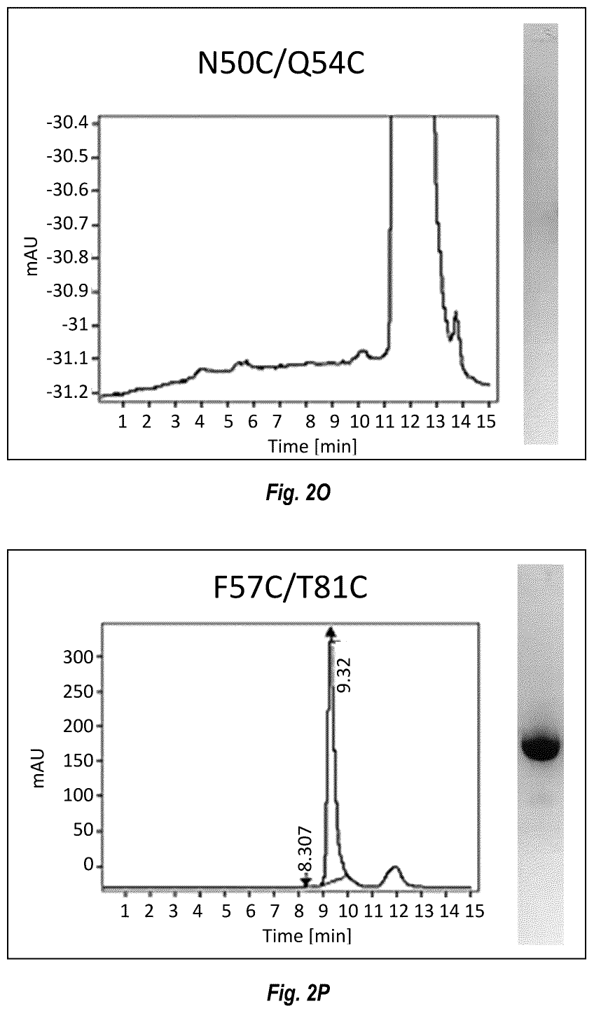

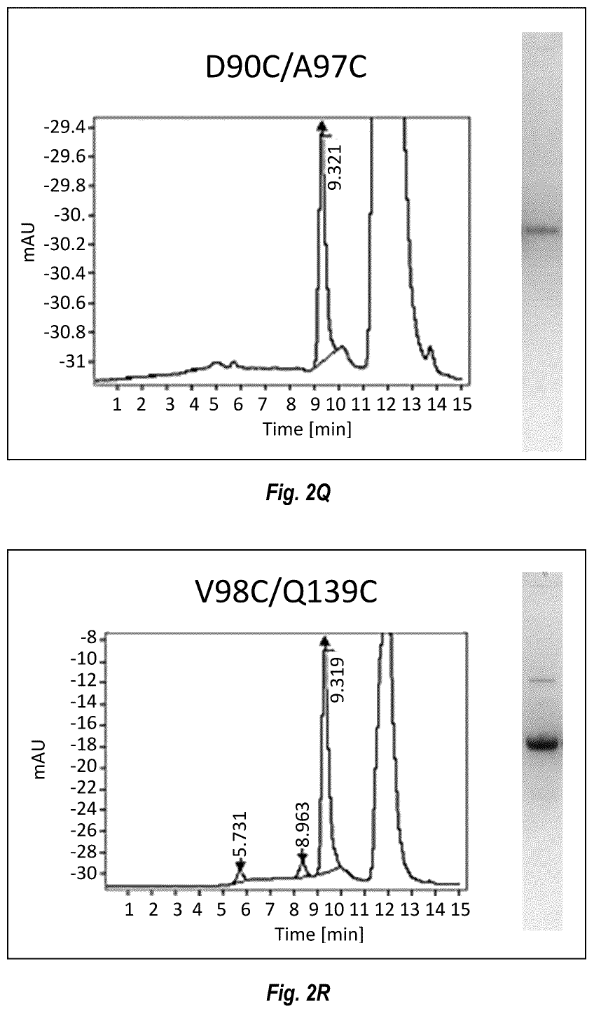

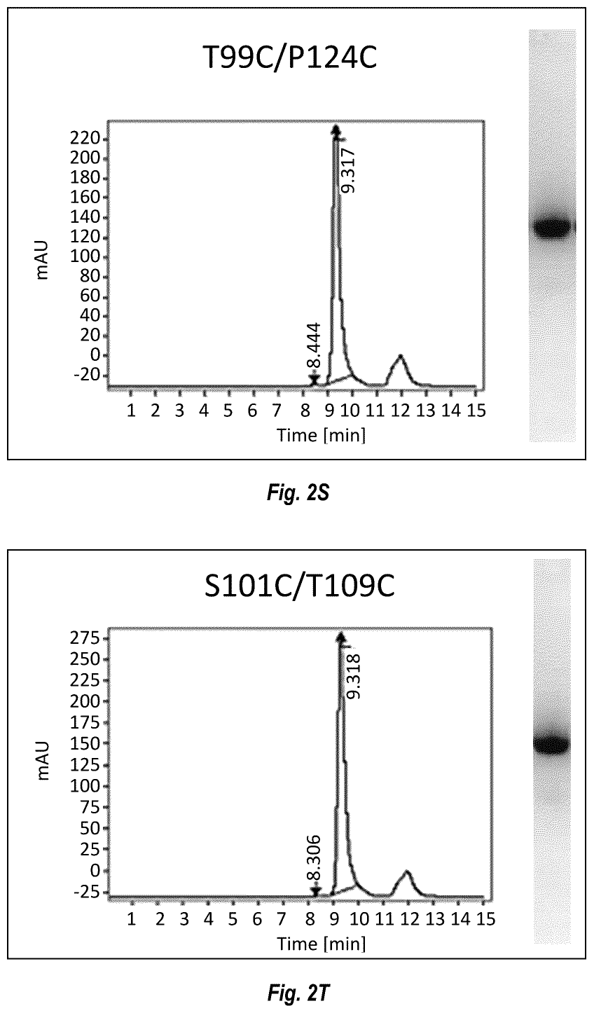

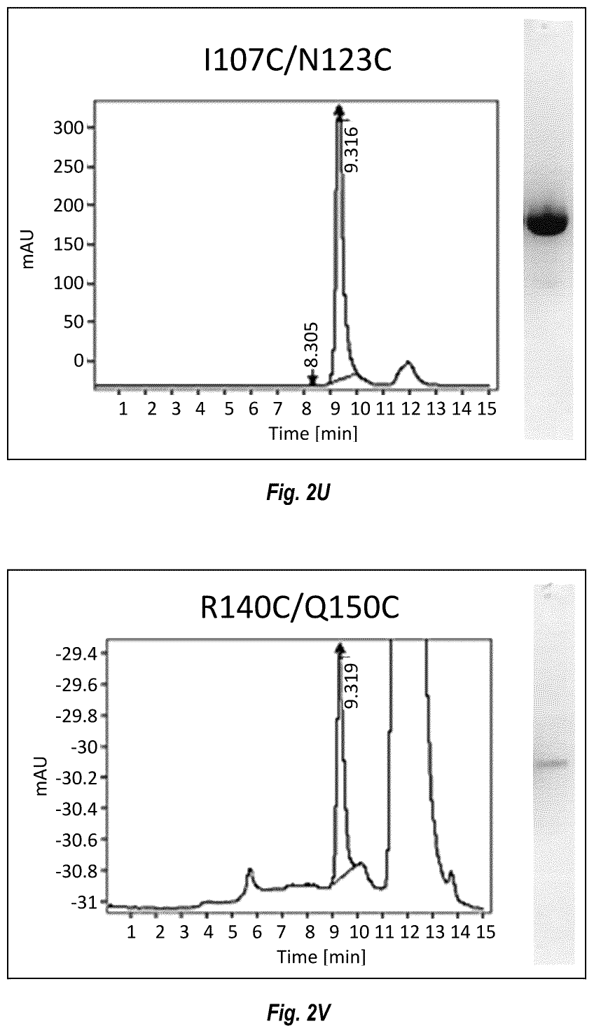

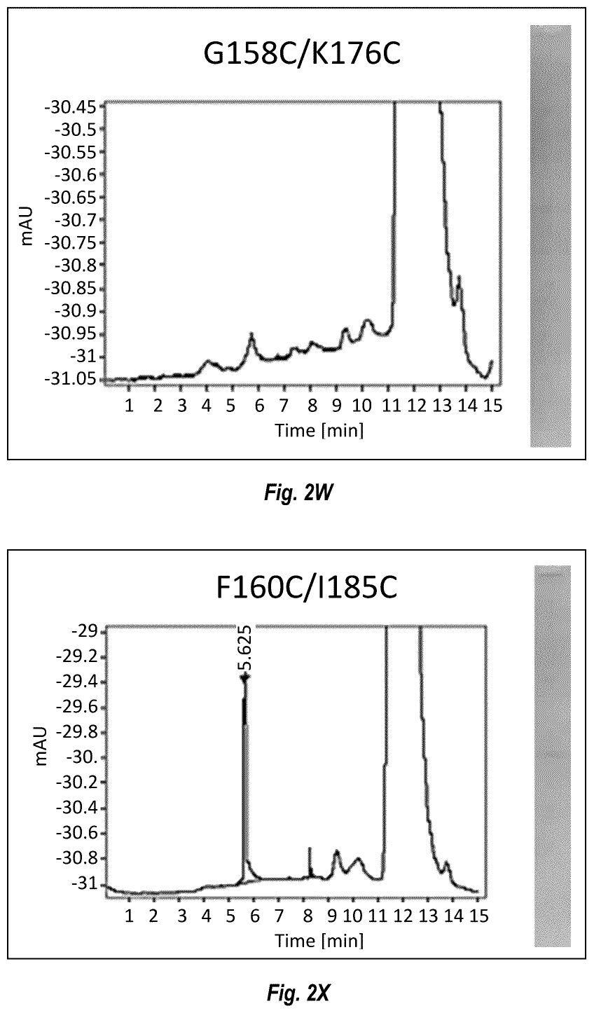

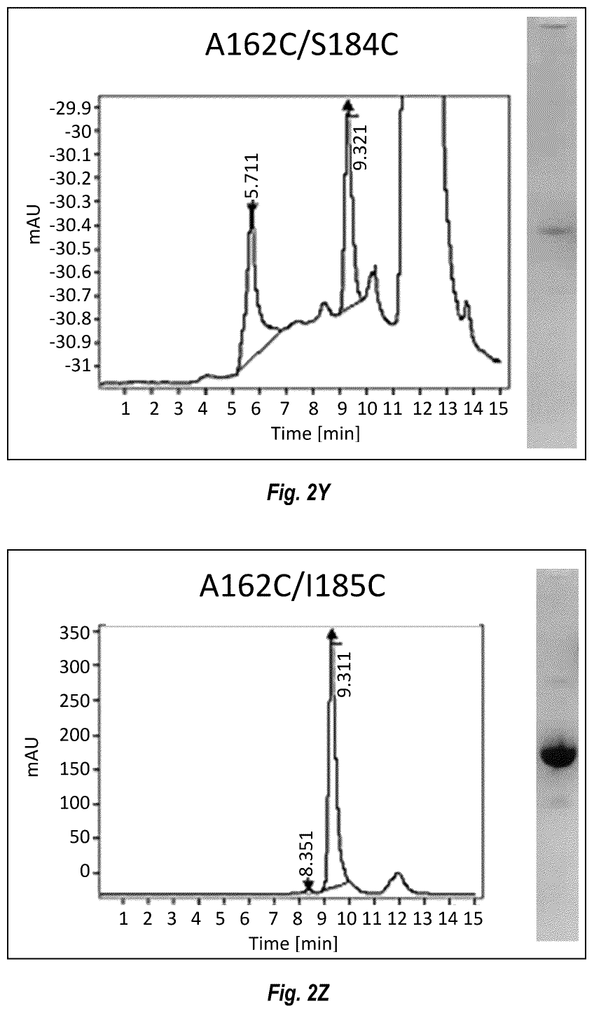

To produce IL-18 in mammalian host cells, an ectopic signal peptide was recombinantly fused to the mature form of IL-18, and a stable fusion partner was fused to the C-terminus—either a monomeric Fc [Ying et al., mAbs, 2014] for human IL-18, or albumin for the mouse ortholog (FIG. 1). Following affinity purification, the wild-type human IL-18 had a high degree of aggregation and lower molecular weight degradation products corresponding to the approximate mass of the Fc tag (FIG. 2A), and no functional IL-18 fusion protein was detected. To reduce the potential liability of the highly-reactive, free native cysteines, constructs were generated in which these cysteines were replaced with serines (“hCS” and “mCS”). While this eliminated aggregation, the production yields were modest and the molecules had a low thermostability (FIG. 2C).

### Example 3: Engineering Disulfide-Stabilized IL-18 Variants

Production yields and thermostability are important quality attributes for a protein therapeutic. To improve IL-18 stability, residue pairs with O-carbon distances within 5A of each other were identified based on IL-18 crystal structures, for potential substitution and formation of disulfide bonds. Native cysteines, if not involved in the intended disulfide bond(s), were substituted with serine. Residue substitutions in human IL-18 variants, including the variant names and respective substitutions relative to WT human IL-18, are shown in Table 2. Residue substitutions in mouse IL-18 variants, including the variant names and respective substitutions relative to WT mouse IL-18, are shown in Table 3.

TABLE 2

| Ortholog | Variant | Substitutions relative to WT IL-18 |
|---|---|---|
| Human | hCS | C74S, C104S, C112S, C163S |
| Human | L45C/E192C | L45C, C74S, C104S, C112S, |
| | | C163S, E192C |
| Human | N50C | N50C, C74S, C104S, C112S |
| Human | L174C | C74S, C104S, C163S, L174C |
| Human | N50C/L174C | N50C, C74S, C104S, L174C |
| Human | Y37C/S91C | Y37C, C74S, S91C, C104S, |
| | | C112S, C163S |
| Human | F38C/I128C | F38C, C74S, C104S, C112S, |
| | | I128C, C163S |
| Human | G39C/K89C | G39C, C74S, K89C, C104S, |
| | | C112S, C163S |
| Human | S43C/S86C | S43C, C74S, S86C, C104S, |
| | | C112S, C163S |
| Human | S46C/V189C | S46C, C74S, C104S, C112S, |
| | | C163S, V189C |
| Human | S46C/I85C | S46C, C74S, I85C, C104S, |
| | | C112S, C163S |
| Human | V47C/Q190C | V47C, C74S, C104S, C112S, |
| | | C163S, Q190C |
| Human | R49C/T188C | R49C, C74S, C104S, C112S, |
| | | C163S, T188C |
| Human | N50C/Q54C | N50C, Q54C, C74S, C104S, |
| | | C112S, C163S |
| Human | F57C/T81C | F57C, C74S, T81C, C104S, |
| | | C112S, C163S |
| Human | D90C/A97C | C74S, D90C, A97C, C104S, |
| | | C112S, C163S |
| Human | V98C/Q139C | C74S, V98C, C104S, C112S, |
| | | Q139C, C163S |
| Human | T99C/P124C | C74S, T99C, C104S, C112S, |
| | | P124C, C163S |
| Human | S101C/T109C | C74S, S101C, C104S, T109C, |
| | | C112S, C163S |
| Human | I107C/N123C | C74S, C104S, I107C, C112S, |
| | | N123C, C163S |
| Human | R140C/Q150C | C74S, C104S, C112S, R140C, |
| | | Q150C, C163S |
| Human | G158C/K176C | C74S, C104S, C112S, G158C, |
| | | C163S, K176C |
| Human | F160C/I185C | C74S, C104S, C112S, F160C, |
| | | C163S, I185C |
| Human | A162C/S184C | C74S, C104S, C112S, A162C, |
| | | C163S, S184C |
| Human | A162C/I185C | C74S, C104S, C112S, A162C, |
| | | C163S, I185C |
| Human | A162C/I185C | C74S, M87K, K89S, Q92L, P93A, |
| | DR Fc* | M96L, C104S, C112S, S141D, |
| | | D146S, N147R, A162C, |
| | | C163S, I185C |

*“DR” refers to “decoy resistant,” and indicates the inclusion of mutations that significantly reduce IL-18BP binding.

TABLE 3

| Ortholog | Variant | Substitutions relative to WT IL-18 |
|---|---|---|
| Mouse | mCS | C42S, C110S, C160S |
| Mouse | T44C/L189C | C42S, T44C, C110S, C160S, L189C |
| Mouse | N49C | C42S, N49C, C110S |
| Mouse | L171C | C42S, C160S, L171C |
| Mouse | N49C/L171C | C42S, N49C, L171C |
| Mouse | N36C/S89C | N36C, C42S, S89C, C110S, C160S |
| Mouse | Y84C | Y84C, C110S, C160S |
| Mouse | T43C/I83C | C42S, T43C, I83C, C110S, C160S |
| Mouse | A45C/L186C | C42S, A45C, C110S, C160S, L186C |
| Mouse | A45C/I83C | C42S, A45C, I83C, C110S, C160S |
| Mouse | F56C/T79C | C42S, F56C, T79C, C110S, C160S |
| Mouse | T97C/P122C | C42S, T97C, C110S, P122C, C160S |
| Mouse | S99C/T107C | C42S, S99C, T107C, C110S, C160S |
| Mouse | M105C/D121C | C42S, M105C, C110S, D121C, C160S |
| Mouse | A159C/V182C | C42S, C110S, A159C, C160S, V182C |

### Example 4: Characterization of Disulfide-Stabilized IL-18 Variants

IL-18 variants produced in either CHO or HTEK293 mammalian host cells were evaluated for expression yields and aggregation levels. The results for human IL-18 are shown in FIG. 2 and Table 4. The results for mouse IL-18 are shown in FIG. 2 and Table 5.

TABLE 4

| | | Yield | Aggregate |
|---|---|---|---|
| Variant | Host | (mg/L) | (%) |
| Human WT | CHO | <10 | 63 |
| hCS | 293 | 5 | 0 |
| L45C/E192C | 293 | 226 | 0 |
| N50C | 293 | 32 | 2 |
| L174C | CHO | 9 | 32 |
| N50C/L174C | CHO | 7 | 16 |
| Y37C/S91C | 293 | 109 | 8 |
| F38C/I128C | 293 | 0 | ND |
| G39C/K89C | 293 | 7 | 82 |
| S43C/S86C | 293 | 239 | <1 |
| S46C/V189C | 293 | 169 | 2 |
| S46C/I85C | 293 | 163 | 2 |
| V47C/Q190C | 293 | 4 | 0 |
| R49C/T188C | 293 | 0 | ND |
| N50C/Q54C | 293 | 0 | ND |
| F57C/T81C | 293 | 316 | <1 |
| D90C/A97C | 293 | 7 | 0 |
| V98C/Q139C | 293 | 28 | 11 |
| T99C/P124C | 293 | 219 | <1 |
| S101C/T109C | 293 | 256 | <1 |
| I107C/N123C | 293 | 280 | <1 |
| R140C/Q150C | 293 | 6 | 0 |
| G158C/K176C | 293 | 0 | ND |
| F160C/I185C | 293 | 5 | 100 |
| A162C/S184C | 293 | 7 | 53 |
| A162C/I185C | 293 | 322 | 2 |

TABLE 5

| Mouse IL-18 | | Yield |
|---|---|---|
| Variants | Host | (mg/L) |
| mCS | 293 | 0.1-1 |
| T44C/L189C | 293 | 35 |
| N49C | 293 | 1-3 |
| L171C | 293 | 0.1-1 |
| N49C/L171C | 293 | 0.1-1 |
| N36C/S89C | 293 | 3-5 |
| A45C/L186C | 293 | 3-5 |
| A45C/I83C | 293 | 1-3 |
| T43C/I83C | 293 | 0.1-1 |
| F56C/T79C | 293 | >10 |
| T97C/P122C | 293 | 3-5 |
| S99C/T107C | 293 | >10 |
| M105C/D121C | 293 | 0.1-1 |
| A159C/V182C | 293 | >10 |

Disulfide-stabilized human and mouse IL-18 variants demonstrated significantly increased yields, and disulfide-stabilized human IL-18 variants demonstrated reduced aggregate levels with respect to WT IL-18. In particular, L45C/E192C had no detectable aggregation after a single-step purification and showed yields that were more than an order of magnitude greater than either human wild-type IL-18 or hCS IL-18 (in which native cysteines were replaced with serines). Increased yield was also observed when comparing the equivalent mouse variant, T44C/L189C, to mCS.

Additionally, the disulfide-stabilized IL-18 variants that were selected for thermostability evaluation by differential scanning fluorimetry (DSF) all had enhanced apparent melting temperatures, under non-reducing conditions, relative to their respective WT IL-18, with L45C/E192C exhibiting greater than 15 degrees Celsius increase in melting temperature relative to human wild-type IL-18 (Table 6).

TABLE 6

| | Non-reduced | Reduced T_{M} |
|---|---|---|
| Variant | T_{M} (° C.) | (° C.) |
| Human WT | 35.8 | 47.8 |
| hCS | 37.8 | 37.3 |
| L45C/E192C | 51.0 | 40.6 |
| N50C | 47.8 | 43.1 |
| L174C | 37.8 | 37.3 |
| N50C/L174C | 47.8 | 41.9 |
| Y37C/S91C | 49.2 | 38.1 |
| S43C/S86C | 54.6 | 38.2 |
| S46C/V189C | 47.6 | 40.4 |
| S46C/I85C | 51.6 | 41.9 |
| F57C/T81C | 59.1 | 56.4 |
| T99C/P124C | 48.0 | 45.7 |
| S101C/T109C | 50.3 | 47.9 |
| I107C/N123C | 52.9 | 36.8 |
| A162C/I185C | 58.6 | 55.3 |
| Mouse WT | 50.5 | 50.3 |
| T44C/L189C | 52.9 | 36.9 |
| A159C/V182C | 54.5 | 46.7 |

Additionally, no free thiols are present in disulfide-stabilized IL-18 variants. The Experimental masses of IL-18 variants in daltons (Da), as measured by LC/MS following the addition of 30 mM methyl methanethiosulfonate (MMTS) are shown in Table 7, below. The conjugation of MMTS results in mass addition increments of 45.99 Da per free thiol. Theoretical mass includes assumption of no free cysteine thiols, for the stabilized variants, but not for Human WT.

TABLE 7

| | Experimental | Theoretical | Theoretical |
|---|---|---|---|
| Variant | Mass | Mass | Mass + MMTS |
| Human WT | 20609.94 | 20425.16 | 20609.12 |
| (+biotin) | | | |
| Human WT | 18401.59 | 18216.71 | 18400.67 |
| hCS | 19118.92 | 19118.49 | N/A |
| L45C/E192C | 19080.80 | 19080.47 | 19172.45 |
| N50C | 19122.01 | 19121.57 | 19213.55 |
| L174C | 19122.97 | 19122.51 | 19214.49 |
| N50C/L174C | 19125.99 | 19125.59 | 19309.55 |
| Y37C/S91C | 19072.96 | 19072.49 | 19164.47 |
| S43C/S86C | 19149.06 | 19148.59 | 19240.57 |
| S46C/V189C | 19136.91 | 19136.54 | 19228.52 |
| S46C/I85C | 19122.99 | 19122.51 | 19214.49 |
| F57C/T81C | 19074.96 | 19074.47 | 19166.45 |
| T99C/P124C | 19125.01 | 19124.53 | 19216.51 |
| S101C/T109C | 19135.10 | 19134.57 | 19226.55 |
| I107C/N123C | 19096.00 | 19095.49 | 19187.47 |
| A162C/I185C | 19139.03 | 19138.51 | 19230.49 |

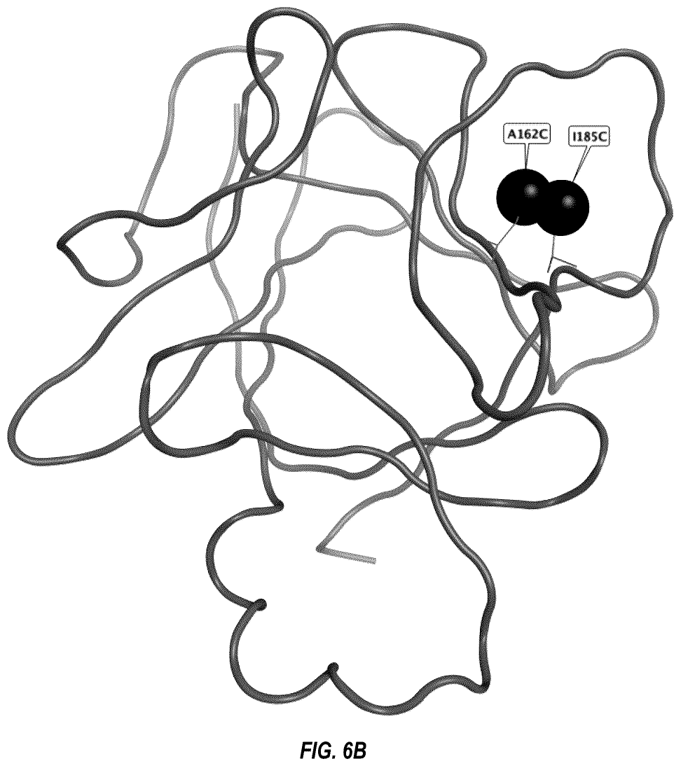

The crystal structures of human L45C/E192C and A162C/185C variants were solved and found to be similar to previously reported structures of WT IL-18. Both structures also confirm the expected novel disulfide formation, consistent with the mass spectrometry data in Table 7. See FIG. 6A-61B.

### Example 5: Binding Kinetics of Disulfide-Stabilized IL-18 Variants

A subset of variants were selected for analyses of binding kinetics to the L-18 receptor, IL-IM, and the decoy receptor, L-18BP, by surface plasmon resonance (SPR) (Table 8). With two exceptions, all kinetics constants are within an order of magnitude of wild-type IL-18. Surprisingly, the N50C and N50C/L174C variants do not have detectable binding to L-18Rα up to 81 nM, yet they retain strong binding to IL-18P.

TABLE 8

| Analyte | Ligand | KD (nM) | Kₐ (M⁻¹s⁻¹) | K_{d} (s⁻¹) |
|---|---|---|---|---|
| Human WT | Human IL-18Rα | 6.6 ± 2.4 | (1.4 ± 0.4) × 10⁵ | (9.4 ± 1.8) × 10⁻³ |
| | Human IL-18BP | 0.041 ± 0.021 | (3.5 ± 1.1) × 10⁵ | (1.5 ± 0.6) × 10⁻⁵ |
| hCS | Human IL-18Rα | 1.7 ± 0.5 | (3.2 ± 0.7) × 10⁴ | (5.6 ± 0.8) × 10⁻⁵ |
| | Human IL-18BP | 0.035 ± 0.035 | (1.4 ± 1.2) × 10⁶ | (4.9 ± 2.5) × 10⁻⁵ |
| Y37C/S91C | Human IL-18Rα | * | * | * |
| | Human IL-18BP | 0.070 ± 0.020 | (2.6 ± 0.7) × 10⁶ | (1.8 ± 0.06) × 10⁻⁴ |
| S43C/S86C | Human IL-18Rα | 54.5 ± 6.0 | (4.4 ± 0.5) × 10⁵ | (2.41 ± 0.08) × 10⁻² |
| | Human IL-18BP | 0.009 ± 0.002 | (3.4 ± 0.5) × 10⁶ | (3.2 ± 0.7) × 10⁻⁵ |
| L45C/E192C | Human IL-18Rα | 58.7 ± 7.0 | (4.7 ± 0.4) × 10⁵ | (2.8 ± 0.2) × 10⁻² |
| | Human IL-18BP | 0.015 ± 0.006 | (2.1 ± 0.8) × 10⁶ | (3.1 ± 0.5) × 10⁻⁵ |
| S46C/I85C | Human IL-18Rα | * | * | * |
| | Human IL-18BP | 0.035 ± 0.009 | (3.3 ± 0.7) × 10⁶ | (1.2 ± 0.2) × 10⁻⁴ |
| S46C/V189C | Human IL-18Rα | 147 ± 31 | (3.6 ± 0.8) × 10⁵ | (5.4 ± 0.2) × 10⁻² |
| | Human IL-18BP | 0.018 ± 0.006 | (2.3 ± 0.3) × 10⁵ | (4.3 ± 1.3) × 10⁻⁵ |
| N50C | Human IL-18Rα | * | * | * |
| | Human IL-18BP | 0.0062 ± 0.0003 | (4.22 ± 0.08) × 10⁶ | (2.6 ± 0.1) × 10⁻⁵ |
| N50C/L174C | Human IL-18Rα | * | * | * |
| | Human IL-18BP | 0.014 ± 0.004 | (3.6 ± 0.6) × 10⁶ | (4.8 ± 0.1) × 10⁻⁵ |
| F57C/T81C | Human IL-18Rα | * | * | * |
| | Human IL-18BP | 0.007 ± 0.002 | (3.4 ± 0.4) × 10⁶ | (2.2 ± 0.4) × 10⁻⁵ |
| T99C/P124C | Human IL-18Rα | 64.4 ± 8.6 | (5.0 ± 0.3) × 10⁵ | (3.2 ± 0.4) × 10⁻² |
| | Human IL-18BP | 0.008 ± 0.002 | (3.0 ± 0.5) × 10⁶ | (2.5 ± 0.3) × 10⁻⁵ |
| S101C/T109C | Human IL-18Rα | 147 ± 22 | (2.8 ± 0.4) × 10⁵ | (4.2 ± 0.3) × 10⁻² |
| | Human IL-18BP | 0.011 ± 0.002 | (2.3 ± 0.3) × 10⁵ | (2.4 ± 0.4) × 10⁻⁵ |
| I107C/N123C | Human IL-18Rα | 14.7 ± 10.2 | (2.1 ± 1.0) × 10⁶ | (3.1 ± 1.5) × 10⁻² |
| | Human IL-18BP | 0.007 ± 0.002 | (2.9 ± 0.6) × 10⁶ | (2.1 ± 0.4) × 10⁻⁵ |
| A162C/I185C | Human IL-18Rα | 68.1 ± 1.4 | (3.11 ± 0.05) × 10⁶ | (2.12 ± 0.02) × 10⁻¹ |
| | Human IL-18BP | 0.0065 ± 0.0003 | (2.91 ± 0.04) × 10⁶ | (1.89 ± 0.08) × 10⁻⁵ |
| L174C | Human IL-18Rα | 32.8 ± 4.1 | (3.5 ± 0.3) × 10⁵ | (1.1 ± 0.1) × 10⁻² |
| | Human IL-18BP | 0.007 ± 0.001 | (2.93 ± 0.09) × 10⁶ | (2.1 ± 0.2) × 10⁻⁵ |
| Mouse WT | Mouse IL-18Rα | 1.27 ± 0.07 | (7.6 ± 0.3) × 10⁶ | (9.7 ± 0.4) × 10⁻³ |
| | Mouse IL-18BP | 0.06 ± 0.02 | (6.5 ± 1.7) × 10⁵ | (4.1 ± 0.8) × 10⁻⁶ |
| T44C/L189C | Mouse IL-18Rα | 5.2 ± 1.0 | (7.6 ± 1.1) × 10⁶ | (4.0 ± 0.5) × 10⁻² |
| | Mouse IL-18BP | 0.005 ± 0.001 | (2.0 ± 0.1) × 10⁶ | (1.1 ± 0.3) × 10⁻⁵ |
| A159C/V182C | Mouse IL-18Rα | 1.11 ± 0.05 | (7.0 ± 0.3) × 10⁶ | (7.7 ± 0.1) × 10⁻³ |
| | Mouse IL-18BP | 0.014 ± 0.002 | (2.23 ± 0.08) × 10⁶ | (3.2 ± 0.4) × 10⁻⁵ |

### Example 6: Potency of Disulfide-Stabilized IL-18 Variants

In order to determine whether the variants have any potency differences relative to WT, their EC50s were evaluated in a native T-cell assay that measured IFNγ production. Production of IFNγ is measured in response to treatments with IL-18 variants.

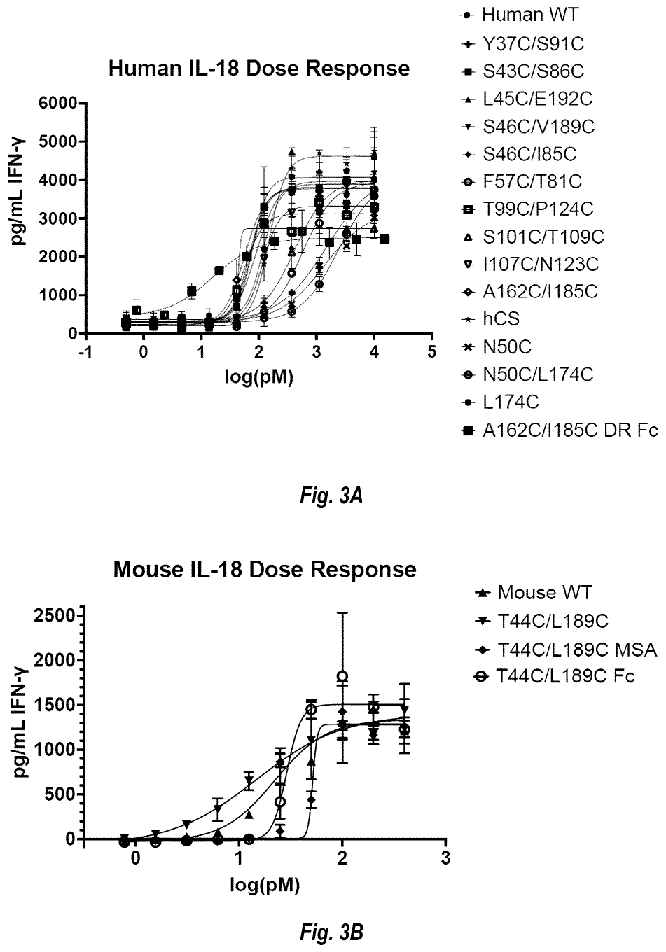

The results of the assay are shown in FIG. 3A-31B and Table 9. Analogous to the observation by SPR, most variants had comparable EC50s to human wild-type IL-18 in the native T-cell assay, with the exception of N50C and N50C/L174C. Y37C/S91C also had significantly reduced potency compared to human wild-type IL-18, and S46C/I85C and F57C/T81C had somewhat reduced potency (FIG. 3A). The N50C-containing variants had more than an order of magnitude reduction in potency (Table 9). Eₘₐₓ, which is the maximum induction of IFNγ expression, was also determined for the IL-18 variants. As shown in FIG. 3A and Table 9, the S101C/T109C variant had a similar EC50 to human wild-type IL-18, but an Emax approximately 30% lower. The remaining variants had Emax values within 20% of human wild-type IL-18. Mouse variants T44C/L189C, T44C/L189C MSA, and T44C/L189C Fc all had EC50s and Emax comparable to mouse wild-type IL-18 (Table 9 and FIG. 3B).

TABLE 9

| | | Eₘₐₓ (pg/mL |
|---|---|---|
| Variant | EC50 (pM) | IFN-γ) |
| Human WT | 117 | 3888 |
| hCS | 151 | 4625 |
| L45C/E192C | 77 | 4070 |
| N50C | 3318 | 3220 |
| L174C | 90 | 3970 |
| N50C/L174C | 2521 | 4069 |
| Y37C/S91C | 1407 | 4208 |
| S43C/S86C | 69 | 3795 |
| S46C/V189C | 73 | 3805 |
| S46C/I85C | 411 | 4006 |
| F57C/T81C | 574 | 3948 |
| T99C/P124C | 55 | 3127 |
| S101C/T109C | 43 | 2739 |
| I107C/N123C | 116 | 3323 |
| A162C/I185C | 59 | 3776 |
| A162C/I185C DR Fc | 16 | 2496 |
| Mouse WT | 22 | 1341 |
| T44C/L189C | 15 | 1401 |
| T44C/L189C MSA | 52 | 1285 |
| T44C/L189C Fc | 29 | 1508 |

### Example 7: Efficacy of Mouse IL-18 T44C/L189C Fc in the MC38 Colon Cancer Model

In the first experiment, C57BL-6 mice were inoculated with 1 million MC38 cells subcutaneously. Treatment was initiated when the tumors achieved a mean tumor volume of approximately 130-230 mm³, which was approximately 7-9 days after inoculation. All groups were dosed twice weekly, for a total of five doses. The mouse IL-18 T44C/L189C Fc (“dsIL-18Fc”) groups were dosed intraperitoneally (IP) at 0.1, 1, or 5 mg/kg. Anti-PD-L1 antibody and the control antibody were each dosed at 10 mg/kg intravenously for the first dose, followed by IP for subsequent doses.

In the second experiment, C57BL-6 mice were inoculated with 0.1 million MC38 cells subcutaneously. Treatment was initiated when the tumors achieved a mean tumor volume of approximately 130-230 mm³, approximately 14-18 days after inoculation. Both IL-18 WT and PBS control groups were dosed twice weekly, for a total of five doses. The IL-18 WT group was dosed IP at 3 mg/kg, which is the molar equivalent of 7.4 mg/kg of dsIL-18Fc.

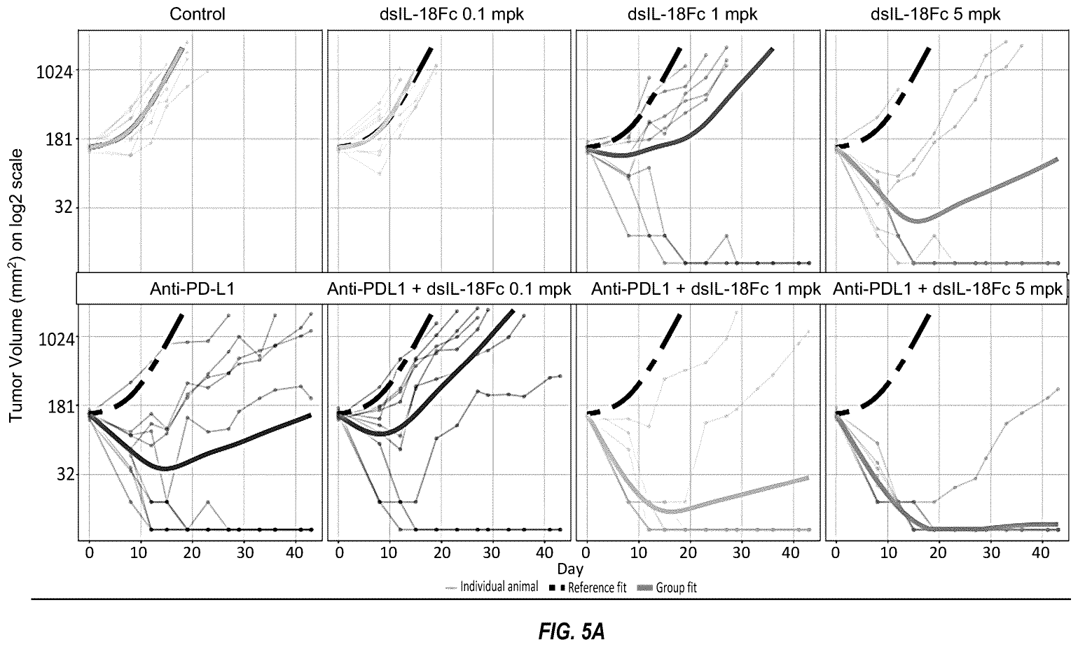

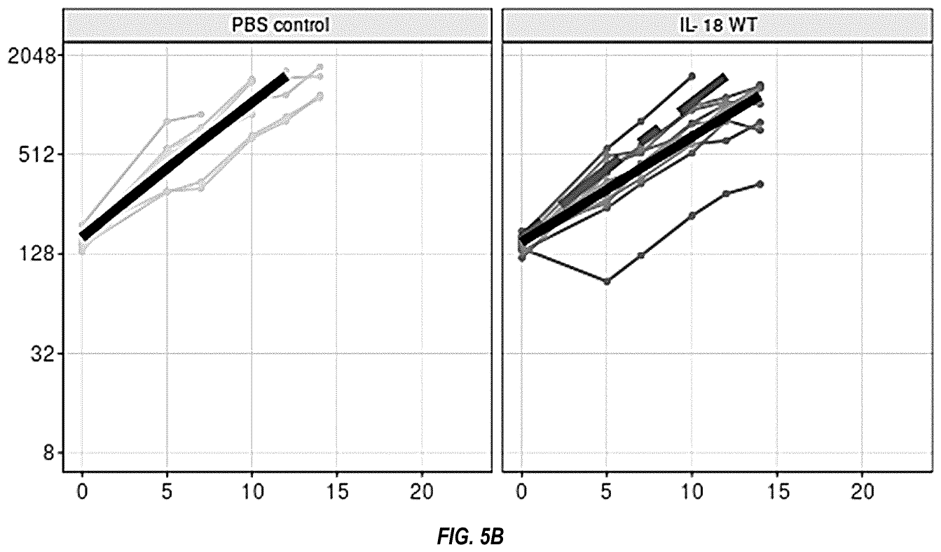

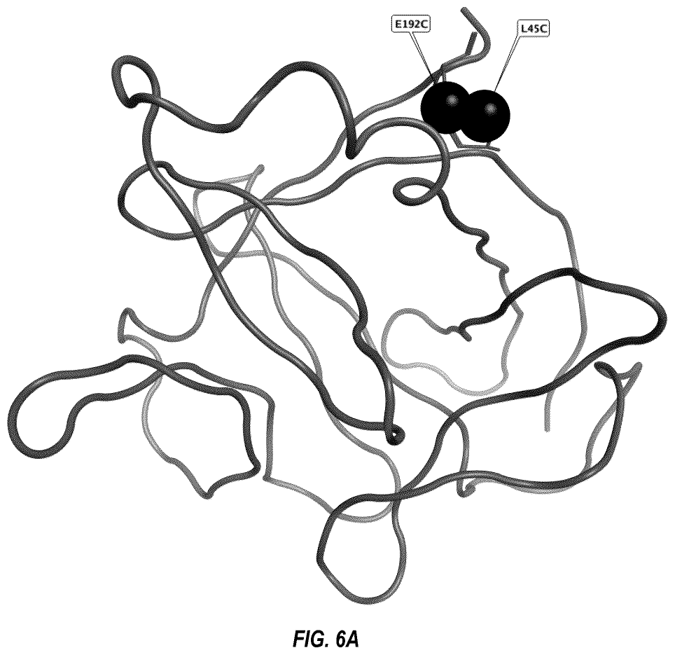

The results are shown in FIGS. 5A and 5B. Surprisingly, while treatment with WT IL-18 at 3 mg/kg did not show meaningful tumor growth inhibition as a single agent (FIG. 5A), treatment with mouse IL-18 T44C/L189C Fc (“dsIL-18Fc”), which has similar potency and affinity for IL-18BP as WT IL-18 (see, e.g, Table 8), at 1 mg/kg and 5 mg/kg resulted in moderate to strong single-agent tumor growth inhibition, which was further enhanced when combined with anti-PD-L1 antibody treatment (FIG. 5B). These results suggest that abrogating IL-18BP binding is not required for efficacy of an IL-18 variant.

### IV. TABLE OF CERTAIN SEQUENCES

| SEQ | | |
|---|---|---|
| ID | | |
| NO | Description | Sequence |
| 1 | Human mature | YFGKLESKLS VIRNLNDQVL FIDQGNRPLF EDMTDSDCRD NAPRTIFIIS |
| | IL-18 | MYKDSQPRGM AVTISVKCEK ISTLSCENKI ISFKEMNPPD NIKDTKSDII |
| | | FFQRSVPGHD NKMQFESSSY EGYFLACEKE RDLFKLILKK EDELGDRSIM |
| | | FTVQNED |
| 2 | Mouse mature | NFGRLHCTTAVIRNINDQVLFVDKROPVFEDMTDIDOSASEPQTRLIIYMYKDSE |
| | IL-18 | VRGLAVTLSVKDSKMSTLSCKNKIISFEEMDPPENIDDIQSDLIFFQKRVPGHNK |
| | | MEFESSLYEGHFLACQKEDDAFKLILKKKDENGDKSVMFTLTNLHQS |
| 4 | Human IL-18 | YFGKLESKLSVIRNLNDQVLFIDQGNRPLFEDMTDSDSRDNAPRTIFIISMYKDS |
| | hCS | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTVQNED |
| 5 | Human IL-18 | YFGKLESKCSVIRNLNDQVLFIDQGNRPLFEDMTDSDSRDNAPRTIFIISMYKDS |
| | L45C/E192C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTVONCD |
| 6 | Human IL-18 | YFGKLESKLSVIRCLNDQVLFIDOGNRPLFEDMTDSDSRDNAPRTIFIISMYKDS |
| | N50C | QPRGMAVTISVKSEKISTLSSENKIISFKEMNPPDNIKDTKSDIIFFQRSVPGHD |
| | | NKMQFESSSYEGYFLACEKERDLFKLILKKEDELGDRSIMFTVQNED |
| 7 | Human IL-18 | YFGKLESKLSVIRNLNDQVLFIDQGNRPLFEDMTDSDSRDNAPRTIFIISMYKDS |
| | L174C | QPRGMAVTISVKSEKISTLSCENKIISFKEMNPPDNIKDTKSDIIFFQRSVPGHD |
| | | NKMQFESSSYEGYFLASEKERDLFKLICKKEDELGDRSIMFTVQNED |
| 8 | Human IL-18 | YFGKLESKLSVIRCLNDQVLFIDQGNRPLFEDMTDSDSRDNAPRTIFIISMYKDS |
| | N50C/L174C | QPRGMAVTISVKSEKISTLSCENKIISFKEMNPPDNIKDTKSDIIFFQRSVPGHD |
| | | NKMQFESSSYEGYFLACEKERDLFKLICKKEDELGDRSIMFTVQNED |
| 9 | Human IL-18 | CFGKLESKLSVIRNLNDQVLFIDQGNRPLFEDMTDSDSRDNAPRTIFIISMYKDC |
| | Y37C/S91C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTVQNED |
| 10 | Human IL-18 | YCGKLESKLSVIRNLNDQVLFIDQGNRPLFEDMTDSDSRDNAPRTIFIISMYKDS |
| | F38C/1128C | QPRGMAVTISVKSEKISTLSSENKIISFKEMNPPDNCKDTKSDIIFFQRSVPGHD |
| | | NKMQFESSSYEGYFLASEKERDLFKLILKKEDELGDRSIMFTVQNED |
| 11 | Human IL-18 | YFCKLESKLSVIRNLNDQVLFIDQGNRPLFEDMTDSDSRDNAPRTIFIISMYCDS |
| | G39C/K89C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTVQNED |
| 12 | Human IL-18 | YFGKLECKLSVIRNLNDQVLFIDQGNRPLFEDMTDSDSRDNAPRTIFIICMYKDS |
| | S43C/S86C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTVQNED |
| 13 | Human IL-18 | YFGKLESKLCVIRNLNDQVLFIDQGNRPLFEDMTDSDSRDNAPRTIFIISMYKDS |
| | S46C/V189C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTCQNED |
| 14 | Human IL-18 | YFGKLESKLCVIRNLNDQVLFIDQGNRPLFEDMTDSDSRDNAPRTIFICSMYKDS |
| | S46C/185C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTVQNED |
| 15 | Human IL-18 | YFGKLESKLSCIRNLNDQVLFIDOGNRPLFEDMTDSDSRDNAPRTIFIISMYKDS |
| | V47C/Q190C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTVCNED |
| 16 | Human IL-18 | YFGKLESKLSVICNLNDQVLFIDOGNRPLFEDMTDSDSRDNAPRTIFIISMYKDS |
| | R49C/T188C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFCVQNED |
| 17 | Human IL-18 | YFGKLESKLSVIRCLNDCVLFIDQGNRPLFEDMTDSDSRDNAPRTIFIISMYKDS |
| | N50C/Q54C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTVQNED |
| 18 | Human IL-18 | YFGKLESKLSVIRNLNDQVLCIDQGNRPLFEDMTDSDSRDNAPRCIFIISMYKDS |
| | F57C/T81C | QPRGMAVTISVKSEKISTLSSENKIISFKEMNPPDNIKDTKSDIIFFQRSVPGHD |
| | | NKMQFESSSYEGYFLASEKERDLFKLILKKEDELGDRSIMFTVQNED |
| 19 | Human IL-18 | YFGKLESKLSVIRNLNDQVLFIDQGNRPLFEDMTDSDSRDNAPRTIFIISMYKCS |
| | D90C/A97C | QPRGMCVTISVKSEKISTLSSENKIISFKEMNPPDNIKDTKSDIIFFQRSVPGHD |
| | | NKMOFESSSYEGYFLASEKERDLFKLILKKEDELGDRSIMFTVONED |
| 20 | Human IL-18 | YFGKLESKLSVIRNLNDQVLFIDQGNRPLFEDMTDSDSRDNAPRTIFIISMYKDS |
| | V98C/Q139C | QPRGMACTISVKSEKISTLSSENKIISFKEMNPPDNIKDTKSDIIFFCRSVPGHD |
| | | NKMQFESSSYEGYFLASEKERDLFKLILKKEDELGDRSIMFTVQNED |
| 21 | Human IL-18 | YFGKLESKLSVIRNLNDQVLFIDQGNRPLFEDMTDSDSRDNAPRTIFIISMYKDS |
| | T99C/P124C | QPRGMAVCISVKSEKISTLSSENKIISFKEMNCPDNIKDTKSDIIFFQRSVPGHD |
| | | NKMQFESSSYEGYFLASEKERDLFKLILKKEDELGDRSIMFTVQNED |
| 22 | Human IL-18 | YFGKLESKLSVIRNLNDQVLFIDOGNRPLFEDMTDSDSRDNAPRTIFIISMYKDS |
| | S101C/T109C | QPRGMAVTICVKSEKISCLSSENKIISFKEMNPPDNIKDTKSDIIFFQRSVPGHD |
| | | NKMQFESSSYEGYFLASEKERDLFKLILKKEDELGDRSIMFTVQNED |
| 23 | Human IL-18 | YFGKLESKLSVIRNLNDQVLFIDQGNRPLFEDMTDSDSRDNAPRTIFIISMYKDS |
| | I107C/N123C | QPRGMAVTISVKSEKCSTLSSENKIISFKEMCPPDNIKDTKSDIIFFQRSVPGHD |
| | | NKMQFESSSYEGYFLASEKERDLFKLILKKEDELGDRSIMFTVQNED |
| 24 | Human IL-18 | YFGKLESKLSVIRNLNDQVLFIDOGNRPLFEDMTDSDSRDNAPRTIFIISMYKDS |
| | R140C/Q150C | QPRGMAVTISVKSEKISTLSSENKIISFKEMNPPDNIKDTKSDIIFFQCSVPGHD |
| | | NKMCFESSSYEGYFLASEKERDLFKLILKKEDELGDRSIMFTVQNED |
| 25 | Human IL-18 | YFGKLESKLSVIRNLNDQVLFIDQGNRPLFEDMTDSDSRDNAPRTIFIISMYKDS |
| | G158C/K176C | QPRGMAVTISVKSEKISTLSSENKIISFKEMNPPDNIKDTKSDIIFFQRSVPGHD |
| | | NKMQFESSSYECYFLASEKERDLFKLILKCEDELGDRSIMFTVQNED |
| 26 | Human IL-18 | YFGKLESKLSVIRNLNDQVLFIDQGNRPLFEDMTDSDSRDNAPRTIFIISMYKDS |
| | F160C/1185C | QPRGMAVTISVKSEKISTLSSENKIISFKEMNPPDNIKDTKSDIIFFQRSVPGHD |
| | | NKMQFESSSYEGYCLASEKERDLFKLILKKEDELGDRSCMFTVQNED |
| 27 | Human IL-18 | YFGKLESKLSVIRNLNDQVLFIDOGNRPLFEDMTDSDSRDNAPRTIFIISMYKDS |
| | A162C/S184C | QPRGMAVTISVKSEKISTLSSENKIISFKEMNPPDNIKDTKSDIIFFQRSVPGHD |
| | | NKMQFESSSYEGYFLCSEKERDLFKLILKKEDELGDRCIMFTVQNED |
| 28 | Human IL-18 | YFGKLESKLSVIRNLNDQVLFIDOGNRPLFEDMTDSDSRDNAPRTIFIISMYKDS |
| | A162C/1185C | QPRGMAVTISVKSEKISTLSSENKIISFKEMNPPDNIKDTKSDIIFFQRSVPGHD |
| | | NKMQFESSSYEGYFLCSEKERDLFKLILKKEDELGDRSCMFTVQNED |
| 30 | Mouse IL-18 | NFGRLHSTTAVIRNINDQVLFVDKRQPVFEDMTDIDOSASEPQTRLIIYMYKDSE |
| | mCS | VRGLAVTLSVKDSKMSTLSSKNKIISFEEMDPPENIDDIQSDLIFFQKRVPGHNK |
| | | MEFESSLYEGHFLASQKEDDAFKLILKKKDENGDKSVMFTLTNLHQS |
| 31 | Mouse IL-18 | NFGRLHSTCAVIRNINDQVLFVDKROPVFEDMTDIDQSASEPQTRLIIYMYKDSE |
| | T44C/L189C | VRGLAVTLSVKDSKMSTLSSKNKIISFEEMDPPENIDDIQSDLIFFQKRVPGHNK |
| | | MEFESSLYEGHFLASQKEDDAFKLILKKKDENGDKSVMFTLTNCHQS |
| 32 | Mouse IL-18 | NFGRLHSTTAVIRCINDQVLFVDKROPVFEDMTDIDOSASEPQTRLIIYMYKDSE |
| | N49C | VRGLAVTLSVKDSKMSTLSSKNKIISFEEMDPPENIDDIQSDLIFFQKRVPGHNK |
| | | MEFESSLYEGHFLACQKEDDAFKLILKKKDENGDKSVMFTLTNLHQS |
| 33 | Mouse IL-18 | NFGRLHSTTAVIRNINDQVLFVDKROPVFEDMTDIDOSASEPQTRLIIYMYKDSE |
| | L171C | VRGLAVTLSVKDSKMSTLSCKNKIISFEEMDPPENIDDIQSDLIFFQKRVPGHNK |
| | | MEFESSLYEGHFLASQKEDDAFKLICKKKDENGDKSVMFTLTNLHQS |
| 34 | Mouse IL-18 | NFGRLHSTTAVIRCINDQVLFVDKROPVFEDMTDIDOSASEPQTRLIIYMYKDSE |
| | N49C/L171C | VRGLAVTLSVKDSKMSTLSCKNKIISFEEMDPPENIDDIQSDLIFFQKRVPGHNK |
| | | MEFESSLYEGHFLACQKEDDAFKLICKKKDENGDKSVMFTLTNLHQS |
| 35 | Human IL-18Rα | AESCTSRPHITVVEGEPFYLKHCSCSLAHEIETTTKSWYKSSGSQEHVELNPRSS |
| | | SRIALHDCVLEFWPVELNDTGSYFFQMKNYTQKWKLNVIRRNKHSCFTERQVTSK |
| | | IVEVKKFFQITCENSYYQTLVNSTSLYKNCKKLLLENNKNPTIKKNAEFEDQGYY |
| | | SCVHFLHHNGKLFNITKTENITIVEDRSNIVPVLLGPKLNHVAVELGKNVRLNCS |
| | | ALLNEEDVIYWMFGEENGSDPNIHEEKEMRIMTPEGKWHASKVLRIENIGESNLN |
| | | VLYNCTVASTGGTDTKSFILVRKADMADIPGHVFTR |
| 36 | Human IL-18BP | TPVSQTTTAATASVRSTKDPCPSQPPVFPAAKQCPALEVTWPEVEVPLNGTLSLS |
| | | CVACSRFPNFSILYWLGNGSFIEHLPGRLWEGSTSRERGSTGTQLCKALVLEQLT |
| | | PALHSTNFSCVLVDPEQVVQRHVVLAQLWAGLRATLPPTQEALPSSHSSPQQQG |
| 37 | Mouse IL-18Rα | KSCIHRSQIHVVEGEPFYLKPCGISAPVHRNETATMRWFKGSASHEYRELNNRSS |
| | | PRVTFHDHTLEFWPVEMEDEGTYISQVGNDRRNWTLNVTKRNKHSCFSDKLVTSR |
| | | DVEVNKSLHITCKNPNYEELIQDTWLYKNCKEISKTPRILKDAEFGDEGYYSCVF |
| | | SVHHNGTRYNITKTVNITVIEGRSKVTPAILGPKCEKVGVELGKDVELNCSASLN |
| | | KDDLFYWSIRKEDSSDPNVQEDRKETTTWISEGKLHASKILRFQKITENYLNVLY |
| | | NCTVANEEAIDTKSFVLVRKEIPDIPGHVFTG |
| 38 | Mouse IL-18BP | TSAPQTTATVLTGSSKDPCSSWSPAVPTKQYPALDVIWPEKEVPLNGTLTLSCTA |
| | | CSRFPYFSILYWLGNGSFIEHLPGRLKEGHTSREHRNTSTWLHRALVLEELSPTL |
| | | RSTNFSCLFVDPGQVAQYHIILAQLWDGLKTAPSPSQETLSSHSPVSRSAGPGVA |
| 39 | Human IL-18 | YFGKLESKLSVIRNLNDQVLFIDOGNRPLFEDMTDSDSRDNAPRTIFIISKYSDS |
| | A162C/1185C | LARGLAVTISVKSEKISTLSSENKIISFKEMNPPDNIKDTKSDIIFFORDVPGHS |
| | DR Fc | RKMQFESSSYEGYFLCSEKERDLFKLILKKEDELGDRSCMFTVQNEDGGGGAGGG |
| | | GAGGGGDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLNGKEYKCKVS |
| | | NKALGAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHEALHN |
| | | HYTQKSLSLSPGK |
| 40 | Mouse IL-18 | NFGRLHSTCAVIRNINDQVLFVDKROPVFEDMTDIDOSASEPQTRLIIYMYKDSE |
| | T44C/L189C Fc | VRGLAVTLSVKDSKMSTLSSKNKIISFEEMDPPENIDDIQSDLIFFQKRVPGHNK |
| | | MEFESSLYEGHFLASQKEDDAFKLILKKKDENGDKSVMFTLTNCHQSGGGGAGGG |
| | | GEPRGPTIKPCPPCKCPAPNAAGGPSVFIFPPKIKDVLMISLSPIVTCVVVDVSE |
| | | DDPDVQISWFVNNVEVHTAQTQTHREDYNSTLRVVSALPIQHQDWMSGKEFKCKV |
| | | NNKDLGAPIERTISKPKGSVRAPQVYVLPPPEEEMTKKQVTLTCMVTDEMPEDIY |
| | | VEWTNNGKTELNYKNTEPVLDSDGSYFMYSKLRVEKKNWVERNSYSCSVVHEGLH |
| | | NHHTTKSFSRTPGK |
| 41 | Mouse IL-18 | NFGRLHSTCAVIRNINDQVLFVDKROPVFEDMTDIDOSASEPQTRLIIYMYKDSE |
| | T44C/L189C MSA | VRGLAVTLSVKDSKMSTLSSKNKIISFEEMDPPENIDDIQSDLIFFQKRVPGHNK |
| | | MEFESSLYEGHFLASQKEDDAFKLILKKKDENGDKSVMFTLTNCHQSGGGENLYF |
| | | QGHHHHHHHHGGGEAHKSEIAHRYNDLGEQHFKGLVLIAFSQYLOKSSYDEHAKL |
| | | VQEVTDFAKTCVADESAANCDKSLHTLFGDKLCAIPNLRENYGELADCCTKOEPE |
| | | RNECFLQHKDDNPSLPPFERPEAEAMCTSFKENPTTFMGHYLHEVARRHPYFYAP |
| | | ELLYYAEQYNEILTOCCAEADKESCLTPKLDGVKEKALVSSVRQRMKCSSMOKFG |
| | | ERAFKAWAVARLSQTFPNADFAEITKLATDLTKVNKECCHGDLLECADDRAELAK |
| | | YMCENQATISSKLQTCCDKPLLKKAHCLSEVEHDTMPADLPAIAADFVEDQEVCK |
| | | NYAEAKDVFLGTFLYEYSRRHPDYSVSLLLRLAKKYEATLEKCCAEANPPACYGT |
| | | VLAEFQPLVEEPKNLVKTNCDLYEKLGEYGFQNAILVRYTQKAPQVSTPTLVEAA |
| | | RNLGRVGTKCCTLPEDQRLPCVEDYLSAILNRVCLLHEKTPVSEHVTKCCSGSLV |
| | | ERRPCFSALTVDETYVPKEFKAETFTFHSDICTLPEKEKQIKKQTALAELVKHKP |
| | | KATAEQLKTVMDDFAQFLDTCCKAADKDTCFSTEGPNLVTRCKDALA |

1. A polypeptide comprising a modified human IL-18 polypeptide, wherein the amino acid sequence of the modified human IL-18 polypeptide is at least 80%, at least 85%, at least 90%, at least 91%, at least 92%, at least 93%, at least 94%, at least 95%, at least 96%, or at least 97% identical to the amino acid sequence of SEQ ID NO: 1, and wherein the modified human IL-18 polypeptide comprises at least one pair of cysteines that are capable of forming a disulfide bond.

2. The polypeptide of claim 1, wherein the modified human IL-18 polypeptide does not comprise free cysteines.

3. The polypeptide of claim 1, wherein the modified human IL-18 polypeptide comprises one or two pairs of cysteines, wherein each pair of cysteines forms a disulfide bond.

4. The polypeptide of claim 3, wherein at least one, at least two, at least three, or all four cysteines in the amino acid sequence of SEQ ID NO: 1 are substituted with another amino acid.

5. (canceled)

6. The polypeptide of claim 1, wherein the modified human IL-18 polypeptide comprises one, two, three, or four of amino acid substitutions C74S, C104S, C112S, and/or C163S, wherein amino acid numbering is according to FIG. 4A.

7. (canceled)

8. A polypeptide comprising a modified human IL-18 polypeptide, wherein the amino acid sequence of the modified human IL-18 polypeptide is at least 80%, at least 85%, at least 90%, at least 91%, at least 92%, at least 93%, at least 94%, at least 95%, at least 96%, or at least 97% identical to the amino acid sequence of SEQ ID NO: 1, and wherein the modified human IL-18 polypeptide comprises a set of amino acid substitutions selected from:
a) L45C and E192C;
b) Y37C and S91C;
c) S43C and S86C;
d) S46C and V189C;
e) S46C and I85C;
f) V47C and Q190C;
g) N50C;
h) N50C and L174C;
i) F57C and T81C;
j) D90C and A97C;
k) V98C and Q139C;
l) T99C and P124C;
m) S101C and T109C;
n) I107C and N123C;
o) R140C and Q150C; and
p) A162C and I185C;
wherein amino acid numbering is according to FIG. 4A.

9. The polypeptide of claim 1, wherein the modified human IL-18 polypeptide comprises a set of amino acid substitutions selected from:
a) L45C and E192C;
b) Y37C and S91C;
c) S43C and S86C;
d) S46C and V189C;
e) S46C and I85C;
f) V47C and Q190C;
g) F57C and T81C;
h) D90C and A97C;
i) V98C and Q139C;
j) T99C and P124C;
k) S101C and T109C;
l) I107C and N123C;
m) R140C and Q150C; and
n) A162C and I185C;
and comprises amino acid substitutions C74S, C104S, C112S, and C163S, wherein amino acid numbering is according to FIG. 4A.

10. The polypeptide of claim 8, wherein the modified human IL-18 polypeptide comprises a set of amino acid substitutions selected from:
a) N50C, C74S, C104S, and C112S; and
b) N50C, C74S, C104S, and L174C;
wherein amino acid numbering is according to FIG. 4A.

11. The polypeptide of claim 8, wherein the amino acid sequence of the modified human IL-18 polypeptide is at least 90%, at least 95%, at least 96%, at least 97%, at least 98%, or at least 99% identical to an amino acid sequence selected from SEQ ID NOs: 5, 6, 8, 9, 12, 13, 15, 18, 19-24, and 27.

12. The polypeptide of claim 8, wherein the modified human IL-18 polypeptide comprises an amino acid sequence selected from SEQ ID NOs: 5, 6, 8, 9, 12, 13, 15, 18, 19-24, and 27.

13. The polypeptide of claim 1, wherein the polypeptide binds IL-18Rα with an affinity of less than 100 nM, or less than 50 nM, or less than 30 nM, or less than 20 nM, less than 10 nM, between 0.1 nM and 100 nM, or between 1 nM and 100 nM, as measured by surface plasmon resonance.

14. (canceled)

15. The polypeptide of claim 1, wherein the polypeptide binds to TL-18Rα with an affinity of greater than 50 nM, greater than 60 nM, greater than 70 nM, greater than 80 nM, greater than 90 nM, greater than 100 nM, between 50 nM and 1 mM, between 60 nM and 1 mM, between 70 nM and 1 mM, between 80 nM and 1 mM, or binds with significantly reduced affinity to IL-18Rα compared to wild-type IL-18, or does not detectably bind IL-18Rα up to 81 nM, as measured by surface plasmon resonance.

16. (canceled)

17. (canceled)

18. The polypeptide of claim 1, wherein the polypeptide binds to IL-18BP with an affinity of less than 1 nM, less than 100 pM, or less than 50 pM, or less than 30 pM, or less than 20 pM, less than 10 pM, between 1 fM and 1 nM, between 10 fM and 1 nM, between 1 fM and 100 pM, between 10 fM and 100 pM, between 1 fM and 50 pM, between 10 fM and 50 pM, between 1 fM and 30 pM, or between 10 fM and 30 pM, as measured by surface plasmon resonance.

19. (canceled)

20. The polypeptide of claim 1, wherein:
(a) the polypeptide induces signaling through the IL-18 receptor in a reporter assay with an EC50 of less than 1 nM, less than 800 pM, less than 700 pM, less than 600 pM, less than 500 pM, less than 400 pM, less than 300 pM, less than 200 pM, less than 100 pM, between 1 pM and 1 nM, between 1 pM and 800 pM, between 1 pM and 500 pM, or between 1 pM and 300 pM; and/or
(b) the polypeptide induces IFNγ expression in human lymphocytes in vitro with an EC50 of less than 1 nM, less than 800 pM, less than 700 pM, less than 600 pM, less than 500 pM, less than 400 pM, less than 300 pM, less than 200 pM, less than 100 pM, between 1 pM and 1 nM, between 1 pM and 800 pM, between 1 pM and 500 pM, or between 1 pM and 300 pM, optionally wherein the lymphocytes are T cells or NK cells.

21. (canceled)

22. (canceled)

23. (canceled)

24. The polypeptide of claim 1, wherein the polypeptide induces IFNγexpression in human lymphocytes in vitro to a substantially reduced extent than wild-type human IL-18, wherein the lymphocytes are T cells or NK cells.

25. (canceled)

26. The polypeptide of claim 1, wherein the modified human IL-18 polypeptide further comprises at least one, at least two, at least three, at least four, at least five, or at least six substitutions at a position selected from Y37, L41, K44, M87, K89, S91, Q92, P93, G95, M96, E113, Q139, S141, D146, N147, M149, V189, and N191, wherein amino acid numbering is according to FIG. 4A.

27. The polypeptide of claim 26, wherein the modified human IL-18 polypeptide further comprises at least one, at least two, at least three, at least four, at least five, or at least six substitutions selected from Y37H, Y37R, L41H, L41I, L41Y, K44Q, K44R, M87T, M87K, M87D, M87N, M87E, M87R, K89R, K89G, K89S, K89T, S91K, S91R, Q92E, Q92A, Q92R, Q92V, Q92G, Q92K, Q92L, P93L, P93G, P93A, P93K, G95T, G95A, M96K, M96Q, M96R, M96L, E113D, Q139E, Q139K, Q139P, Q139A, Q139R, S141R, S141D, S141K, S141N, S141A, D146H, D146K, D146N, D146Q, D146E, D146S, D146G, N147H, N147Y, N147D, N147R, N147S, N147G, M149V, M149R, M149T, M149K, V189I, V189T, V189A, N191K, and N191H.

28. The polypeptide of claim 1, wherein the modified human IL-18 polypeptide further comprises substitutions at positions M87, M96, S141, D146, and N147; or at positions M87, K89, Q92, S141, and N147, wherein amino acid numbering is according to FIG. 4A.

29. The polypeptide of claim 28, wherein the modified human IL-18 polypeptide further comprises substitutions (i) M87T or M87K; (ii) M96K or M96L; (iii) S141D, S141N, or S141A; (iv) D146K, D146N, D146S, or D146G; and (v) N147Y, N147Y, N147R, or N147G; or further comprises substitutions (i) M87K; (ii) K89G or K89S; (iii) Q92G, Q92R, or Q92L; (iv) D146N, D146S, or D146G; and (v) N147R or N147G.

30. The polypeptide of claim 1, wherein the modified human IL-18 polypeptide further comprises at least one, at least two, at least three, at least four, at least five, or at least six substitutions at a position selected from Y37, L41, D53, E67, T70, D71, S72, D73, D76, N77, M87, Q91, M96, Q139, H145, M149, and R167, wherein amino acid numbering is according to FIG. 4A.

31. The polypeptide of claim 30, wherein the modified human IL-18 polypeptide further comprises at least one, at least two, at least three, at least four, at least five, or at least six substitutions selected from Y37D, Y37F, Y37H, Y37L, L41F, L41H, D53A, D53G, D53R, D53H, E67A, E67T, E67G, E67K, E67R, T70A, T70K, T70E, D71S, D71A, D71Y, S72N, S72K, S72R, D73P, D73A, D73R, D73H, D73L, D73V, D76Y, D76S, D76A, N77K, N77S, N77R, M87F, M87L, M87I, Q91H, M96L, M96F, M96I, Q139L, Q139I, H145A, H145P, H145D, M149L, M149I, M149F, and R167S.

32. The polypeptide of claim 1, wherein the modified human IL-18 polypeptide further comprises substitutions D53G, E66A, and either Q139L or Q139I.

33. The polypeptide of claim 32, wherein the modified human IL-18 polypeptide further comprises substitutions D71S and M87F.

34. The polypeptide of claim 1, wherein the polypeptide comprises a fusion partner.

35. (canceled)

36. The polypeptide of claim 34, wherein the fusion partner is an Fc domain, human serum albumin, or an antigen-binding domain.

37. The polypeptide of claim 36, wherein the Fc domain is an IgG1, IgG2, or IgG4 Fc domain.

38. The polypeptide of claim 1, wherein the polypeptide does not comprise a fusion partner.

39. A conjugate comprising the polypeptide of claim 1 and a conjugate moiety.

40. The conjugate of claim 39, wherein the conjugate moiety is a polymer, such as polyethylene glycol (PEG).

41. An isolated nucleic acid encoding the polypeptide of claim 1.

42. A host cell comprising the nucleic acid of claim 41.

43. A host cell that expresses the polypeptide of claim 1.

44. A method of producing a polypeptide comprising a modified human IL-18 polypeptide, comprising culturing the host cell of claim 43 under conditions suitable for the expression of the polypeptide.

45. (canceled)

46. (canceled)

47. (canceled)

48. (canceled)

49. (canceled)

50. A pharmaceutical composition comprising the polypeptide of claim 1, and a pharmaceutically acceptable carrier.

51.-65. (canceled)

66. A method of treating a subject with cancer, comprising administering to the subject an effective amount of the polypeptide of claim 1.

67.-76. (canceled)

77. A method of activating the IL-18 receptor on a cell, comprising contacting the cell with the polypeptide of claim 1.

78. A method of inducing IFNγ expression in a lymphocyte, comprising contacting the lymphocyte with the polypeptide of claim 1.

79. A method of activating a lymphocyte, comprising contacting the lymphocyte with the polypeptide of claim 1.

80. (canceled)

81. (canceled)

82. (canceled)

83. A method of improving the stability of a polypeptide comprising a human IL-18 amino acid sequence, comprising introducing at least one pair of cysteines that form a disulfide bond into the IL-18 amino acid sequence, to make a polypeptide comprising a modified human IL-18 polypeptide.

84. (canceled)

85. (canceled)

86. (canceled)

87. (canceled)

88. (canceled)

89. A method of detecting IL-18BP or IL-18Rα in a sample, comprising contacting the sample with a polypeptide of claim 1, and detecting binding of the polypeptide to IL-18BP or IL-18Rα.

90. (canceled)

91. (canceled)
